SEQUENCE LISTING (114 sequences)

SEQ ID NO: 1 - DNA, 28 bp, Artificial Sequence
  Primer for generating mutants for HBEGF
gaccggaaagtccgtttgcaagaggcag

SEQ ID NO: 2 - DNA, 26 bp, Artificial Sequence
  Primer for generating mutants for HBEGF
ctagccctctccgccgctccaggctc

SEQ ID NO: 3 - DNA, 28 bp, Artificial Sequence
  Primer for generating mutants for HBEGF
ctgcctcttgcaaacggactttccggtc

SEQ ID NO: 4 - DNA, 28 bp, Artificial Sequence
  Primer for generating mutants for HBEGF
gcaagaggcagatctgcttttgagagtc

SEQ ID NO: 5 - DNA, 28 bp, Artificial Sequence
  Primer for generating mutants for HBEGF
gactctcaaaagcagatctgcctcttgc

SEQ ID NO: 6 - DNA, 28 bp, Artificial Sequence
  Primer for generating mutants for HBEGF
cggaaatacaaggactgcatccatggag

SEQ ID NO: 7 - DNA, 28 bp, Artificial Sequence
  Primer for generating mutants for HBEGF
ctccatggatgcagtccttgtatttccg

SEQ ID NO: 8 - DNA, 31 bp, Artificial Sequence
  Primer for generating mutants for HBEGF
ggacttctgcatccatgaatgcaaatatgtg

SEQ ID NO: 9 - DNA, 31 bp, Artificial Sequence
  Primer for generating mutants for HBEGF
cacatatttgcattcatggatgcagaagtcc

SEQ ID NO: 10 - DNA, 29 bp, Artificial Sequence
  Primer for generating mutants for HBEGF
gaatgcaaatatgtggagctccgggctcc

SEQ ID NO: 11 - DNA, 29 bp, Artificial Sequence
  Primer for generating mutants for HBEGF
ggagcccggagctccacatatttgcattc

SEQ ID NO: 12 - DNA, 26 bp, Artificial Sequence
  Primer for generating mutants for HBEGF
atgtgaaggagcgggctccctcctgc

SEQ ID NO: 13 - DNA, 26 bp, Artificial Sequence
  Primer for generating mutants for HBEGF
gcaggagggagcccgctccttcacat

SEQ ID NO: 14 - DNA, 27 bp, Artificial Sequence
  Primer for generating mutants for HBEGF
gctccctcctgctgccacccgggttac

SEQ ID NO: 15 - DNA, 27 bp, Artificial Sequence
  Primer for generating mutants for HBEGF
gtaacccgggtggcagcaggagggagc

SEQ ID NO: 16 - DNA, 25 bp, Artificial Sequence
  Primer for generating mutants for HBEGF
ccctcctgcatccacccgggttacc

SEQ ID NO: 17 - DNA, 25 bp, Artificial Sequence
  Primer for generating mutants for HBEGF
ggtaacccgggtggatgcaggaggg

SEQ ID NO: 18 - DNA, 29 bp, Artificial Sequence
  Primer for generating mutants for HBEGF
ctgccacccgggtcatggagagaggtgtc

SEQ ID NO: 19 - DNA, 29 bp, Artificial Sequence
  Primer for generating mutants for HBEGF
gacacctctctccatgacccgggtggcag

SEQ ID NO: 20 - DNA, 28 bp, Artificial Sequence
  Primer for generating mutants for HBEGF
ccgggttaccatggaaggtgtcatgggc

SEQ ID NO: 21 - DNA, 28 bp, Artificial Sequence
  Primer for generating mutants for HBEGF
gcccatgacaccttccatggtaacccgg

SEQ ID NO: 22 - DNA, 30 bp, Artificial Sequence
  Primer for generating mutants for HBEGF
gcctcccagtggaacgcttatatacctatg

SEQ ID NO: 23 - DNA, 30 bp, Artificial Sequence
  Primer for generating mutants for HBEGF
cataggtatataagcgttccactgggaggc

SEQ ID NO: 24 - DNA, 32 bp, Artificial Sequence
  Primer for generating mutants for HBEGF
cctcccagtggaaaatttatatacctatgacc

SEQ ID NO: 25 - DNA, 32 bp, Artificial Sequence
  Primer for generating mutants for HBEGF
ggtcataggtatataaattttccactgggagg

SEQ ID NO: 26 - DNA, 20 bp, Artificial Sequence
  Forward primer for amplifying sgRNA oligos targeting drug-associated proteins
ttgtggaaaggacgaaaccg

SEQ ID NO: 27 - DNA, 20 bp, Artificial Sequence
  Reverse primer for amplifying sgRNA oligos targeting drug-associated proteins
tgctgtctctagctctacgt

SEQ ID NO: 28 - DNA, 20 bp, Artificial Sequence
  Forward primer for amplifying sgRNA oligos targeting toxin receptors
tcttcatatcgtatcgtgcg

SEQ ID NO: 29 - DNA, 20 bp, Artificial Sequence
  Reverse primer for amplifying sgRNA oligos targeting toxin receptors
tagtcgctaggctataacgt

SEQ ID NO: 30 - DNA, 19 bp, Artificial Sequence
  Primer for cDNA amplification
aacagcatcggagcggaaa

SEQ ID NO: 31 - DNA, 21 bp, Artificial Sequence
  Primer for cDNA amplification
tgggctttatcaccactcctc

SEQ ID NO: 32 - DNA, 21 bp, Artificial Sequence
  Primer for cDNA amplification
aataaaggacccgcgaggaag

SEQ ID NO: 33 - DNA, 21 bp, Artificial Sequence
  Primer for cDNA amplification
ttttcaggagtgtgctgtccg

SEQ ID NO: 34 - DNA, 18 bp, Artificial Sequence
  Primer for cDNA amplification
tcccagctcccaggactc

SEQ ID NO: 35 - DNA, 20 bp, Artificial Sequence
  Primer for cDNA amplification
gggtgttctgagtgtgcagt

SEQ ID NO: 36 - DNA, 21 bp, Artificial Sequence
  Primer for cDNA amplification
agagagccactgtgtggatgc

SEQ ID NO: 37 - DNA, 20 bp, Artificial Sequence
  Primer for cDNA amplification
ggaagtgtgctcgccgtcag

SEQ ID NO: 38 - DNA, 19 bp, Artificial Sequence
  Primer for cDNA amplification
gggctcgtgctgttctcac

SEQ ID NO: 39 - DNA, 20 bp, Artificial Sequence
  Primer for cDNA amplification
gcaccaggcatggaagcaat

SEQ ID NO: 40 - DNA, 20 bp, Artificial Sequence
  Primer for cDNA amplification
cgaaagtgactggtgcctcg

SEQ ID NO: 41 - DNA, 20 bp, Artificial Sequence
  Primer for cDNA amplification
ggtcccaatggcagatccct

SEQ ID NO: 42 - DNA, 19 bp, Artificial Sequence
  Primer for cDNA amplification
aggcgaacctctcggcttt

SEQ ID NO: 43 - DNA, 19 bp, Artificial Sequence
  Primer for cDNA amplification
caatccgcccaaagggaac

SEQ ID NO: 44 - DNA, 20 bp, Artificial Sequence
  Primer for cDNA amplification
ctctgctcggatcgaggtct

SEQ ID NO: 45 - DNA, 19 bp, Artificial Sequence
  Primer for cDNA amplification
gatgcaggtgggagtgagg

SEQ ID NO: 46 - DNA, 20 bp, Artificial Sequence
  Primer for cDNA amplification
ttccccgacccccttcagtg

SEQ ID NO: 47 - DNA, 21 bp, Artificial Sequence
  Primer for cDNA amplification
aggatgggtcactgtgtccgt

SEQ ID NO: 48 - DNA, 17 bp, Artificial Sequence
  Primer for cDNA amplification
tggccgacctcacttcc

SEQ ID NO: 49 - DNA, 25 bp, Artificial Sequence
  Primer for cDNA amplification
aagtaaaacaaatagtcacctctgc

SEQ ID NO: 50 - DNA, 20 bp, Artificial Sequence
  sgRNA sequence for the validation of critical mutation
gtaagcacccgctgtagccc

SEQ ID NO: 51 - DNA, 20 bp, Artificial Sequence
  sgRNA sequence for the validation of critical mutation
ctatcaccttcttcaccgtc

SEQ ID NO: 52 - DNA, 20 bp, Artificial Sequence
  sgRNA sequence for the validation of critical mutation
cctgctaggcaccatggctg

SEQ ID NO: 53 - DNA, 21 bp, Artificial Sequence
  sgRNA sequence for the validation of critical mutation
aatccgctgcgcccccagcca

SEQ ID NO: 54 - DNA, 20 bp, Artificial Sequence
  sgRNA sequence for the validation of critical mutation
gcgcagcggattgcagcttc

SEQ ID NO: 55 - DNA, 20 bp, Artificial Sequence
  sgRNA sequence for the validation of critical mutation
tctgggaacggctgttggct

SEQ ID NO: 56 - DNA, 20 bp, Artificial Sequence
  sgRNA sequence for the validation of critical mutation
tccagccatcctcccgcacg

SEQ ID NO: 57 - DNA, 20 bp, Artificial Sequence
  sgRNA sequence for the validation of critical mutation
tcttagctgactacgcgtaa

SEQ ID NO: 58 - DNA, 20 bp, Artificial Sequence
  sgRNA sequence for the validation of critical mutation
gtccgagatctcgaagcact

SEQ ID NO: 59 - DNA, 20 bp, Artificial Sequence
  sgRNA sequence for the validation of critical mutation
cagcgacactcaccctccgg

SEQ ID NO: 60 - DNA, 20 bp, Artificial Sequence
  sgRNA sequence for the validation of critical mutation
ccttttcctgaatgaagatc

SEQ ID NO: 61 - DNA, 119 bp, Artificial Sequence
  ssODN donor encoded one amino acid substitution for a validated residue
tttttgtggtcttatgtggcctgttttgtgttttcctctgatcttaacagttccgccatggagtcatagttgcagctgacagcaacgctacagcgggtgcttacattgcctcccagacg

SEQ ID NO: 62 - DNA, 119 bp, Artificial Sequence
  ssODN donor encoded one amino acid substitution for a validated residue
tttttgtggtcttatgtggcctgttttgtgttttcctctgatcttaacagttccgccatggagtcatagttgcagctgacagcagggctgccgcgggtgcttacattgcctcccagacg

SEQ ID NO: 63 - DNA, 119 bp, Artificial Sequence
  ssODN donor encoded one amino acid substitution for a validated residue
tttcctctgatcttaacagttccgccatggagtcatagttgcagctgactccagggctacagcgggtgcttacattgcctcacagacggccaagaaggtgatagagatcaacccatacc

SEQ ID NO: 64 - DNA, 119 bp, Artificial Sequence
  ssODN donor encoded one amino acid substitution for a validated residue
agatgcgttccttatttcgaagctcatagattcgacattgccgagccaacagccgttcccagaagctgcaatccgctgcgccgccagcgatggtgcctagcaggtatgggttgatctct

SEQ ID NO: 65 - DNA, 119 bp, Artificial Sequence
  ssODN donor encoded one amino acid substitution for a validated residue
actccagggctacagcgggtgcttacattgcctcccagacggtgaagaaggtgatagagatcaacccatacctgctaggcacaatggctgggggcaccgcggattgcagcttctgggaa

SEQ ID NO: 66 - DNA, 119 bp, Artificial Sequence
  ssODN donor encoded one amino acid substitution for a validated residue
cagtttggaggcagctgctacagagatgcgttccttatttcgaagctcatagattcgacattgccgagccaacagccgttcccagaagctgcaggccgctgcgcccccagccatggtgc

SEQ ID NO: 67 - DNA, 119 bp, Artificial Sequence
  ssODN donor encoded one amino acid substitution for a validated residue
cagtttggaggcagctgctacagagatgcgttccttatttcgaagctcatagattcgacattgccgagccaacagccgttcccagaagctggcatccgctgcgcccccagccatggtgc

SEQ ID NO: 68 - DNA, 119 bp, Artificial Sequence
  ssODN donor encoded one amino acid substitution for a validated residue
atacaccatgttggcaagcagtttggaggcagctgctacagagatgcgttccttatttcgaagctcatagattcggaattggcgagccaacagccgttcccagaagctgcaatccgctg

SEQ ID NO: 69 - DNA, 119 bp, Artificial Sequence
  ssODN donor encoded one amino acid substitution for a validated residue
gcaggcctatgatctggcccgtcgagccatctaccaagccacctacagagatgcctactcaggaggtgcagtcaacctctatcacgtgcgggaggatgactggatccgagtctccagtg

SEQ ID NO: 70 - DNA, 119 bp, Artificial Sequence
  ssODN donor encoded one amino acid substitution for a validated residue
cgcagcctcgcccaccagcacgtcgtaggattccacggctttttcgaggacaacgacttcgtgttcgtggtgttggagctctgtagcagggtgagtgtcgctgctggggaactggaact

SEQ ID NO: 71 - DNA, 119 bp, Artificial Sequence
  ssODN donor encoded one amino acid substitution for a validated residue
aagagatcccggaggtcctagtggacccacgcagccggcggcgctatgtgcggggccgctttttgggcaagggcggctttgcaaaggtgttcgagatctcggacgcggacaccaaggag

SEQ ID NO: 72 - DNA, 119 bp, Artificial Sequence
  ssODN donor encoded one amino acid substitution for a validated residue
cagcctcgcccaccagcacgtcgtaggattccacggctttttcgaggacaacgacttcgtgttcgtggtgttggagctctgtaggcggggcgtgagtgtcgctgctggggaactggaac

SEQ ID NO: 73 - DNA, 119 bp, Artificial Sequence
  ssODN donor encoded one amino acid substitution for a validated residue
ctcccagcctcctccaaattccagcctcttgtagtgatgtcaagcacccctgcaggctcagcaactcacctattttcacctcgagatcttcattcagcagaaggttgcccagcttgagg

SEQ ID NO: 74 - DNA, 119 bp, Artificial Sequence
  ssODN donor encoded one amino acid substitution for a validated residue
actccagggctacagcgggtgcttacattgcctcccagacggtgaagaaggtgatagagatcaacccatacctgctaggcacaatggctgggggcgcggattgcagcttctgggaacgg

SEQ ID NO: 75 - DNA, 24 bp, Artificial Sequence
  Primer for amplification of mutated loci in PSMB5 gene
gtgtttttgtggtcttatgtggcc

SEQ ID NO: 76 - DNA, 23 bp, Artificial Sequence
  Primer for amplification of mutated loci in PSMB5 gene
catgtggttgcagcttaactcac

SEQ ID NO: 77 - DNA, 22 bp, Artificial Sequence
  Primer for amplification of mutated loci in PSMB5 gene
gatgtgaagctcgggtgacatt

SEQ ID NO: 78 - DNA, 23 bp, Artificial Sequence
  Primer for amplification of mutated loci in PSMB5 gene
tcagcattgacaccaagcccttt

SEQ ID NO: 79 - DNA, 25 bp, Artificial Sequence
  Primer for amplification of mutated loci in PSMB5 gene
ctgctaacctcatctccctttccag

SEQ ID NO: 80 - DNA, 23 bp, Artificial Sequence
  Primer for amplification of mutated loci in PSMB5 gene
caagcagctgcatccaccctctt

SEQ ID NO: 81 - DNA, 24 bp, Homo sapiens
gcagctgactccagggctacagcg

SEQ ID NO: 82 - DNA, 24 bp, Homo sapiens
  misc_feature (19)..(19): n is a, c, g, or t
  misc_feature (21)..(21): n is a, c, g, or t
gcagctgactccagggctncngcg

SEQ ID NO: 83 - DNA, 24 bp, Homo sapiens
attgcctcccagacggtgaagaag

SEQ ID NO: 84 - DNA, 24 bp, Homo sapiens
ctaggcaccatggctgggggcgca

SEQ ID NO: 85 - DNA, 24 bp, Homo sapiens
  misc_feature (12)..(12): n is a, c, g, or t
ctaggcaccatngctgggggcgca

SEQ ID NO: 86 - DNA, 24 bp, Homo sapiens
ggcaccatggctgggggcgcagcg

SEQ ID NO: 87 - DNA, 24 bp, Homo sapiens
  misc_feature (19)..(19): n is a, c, g, or t
  misc_feature (21)..(21): n is a, c, g, or t
ggcaccatggctgggggcncngcg

SEQ ID NO: 88 - DNA, 24 bp, Homo sapiens
ctgttggctcggcaatgtcgaatc

SEQ ID NO: 89 - DNA, 24 bp, Homo sapiens
  misc_feature (17)..(17): n is a, c, g, or t
ctgttggctcgccaatnccgaatc

SEQ ID NO: 90 - DNA, 24 bp, Homo sapiens
ctctaccacgtgcgggaggatggc

SEQ ID NO: 91 - DNA, 24 bp, Homo sapiens
  misc_feature (6)..(6): n is a, c, g, or t
  misc_feature (23)..(23): n is a, c, g, or t
ctctancacgtgcgggaggatgnc

SEQ ID NO: 92 - protein, 8 aa, Homo sapiens
AADSRATA

SEQ ID NO: 93 - protein, 8 aa, Homo sapiens
  VARIANT (5)..(5): Xaa can be N or S
AADSXATA

SEQ ID NO: 94 - protein, 8 aa, Homo sapiens
AADSRAAA

SEQ ID NO: 95 - protein, 8 aa, Homo sapiens
IASQTVKK

SEQ ID NO: 96 - protein, 8 aa, Homo sapiens
  VARIANT (6)..(6): Xaa can be A or L
IASQTXKK

SEQ ID NO: 97 - protein, 8 aa, Homo sapiens
LGTMAGGA

SEQ ID NO: 98 - protein, 8 aa, Homo sapiens
  VARIANT (4)..(4): Xaa can be I or V
LGTXAGGA

SEQ ID NO: 99 - protein, 8 aa, Homo sapiens
GTMAGGAA

SEQ ID NO: 100 - protein, 8 aa, Homo sapiens
GTMAGGTA

SEQ ID NO: 101 - protein, 8 aa, Homo sapiens
LLARQCRI

SEQ ID NO: 102 - protein, 8 aa, Homo sapiens
LLARQFRI

SEQ ID NO: 103 - protein, 8 aa, Homo sapiens
LYHVREDG

SEQ ID NO: 104 - protein, 8 aa, Homo sapiens
LYHVREDD

SEQ ID NO: 105 - DNA, 33 bp, Homo sapiens
tgactccagggctacagcgggtgcttacattgc

SEQ ID NO: 106 - DNA, 33 bp, Homo sapiens
  mutation (5)..(6): substitutions
  mutation (8)..(10): substitutions
tgacagcaacgctacagcgggtgcttacattgc

SEQ ID NO: 107 - DNA, 33 bp, Homo sapiens
gcctcccagacggtgaagaaggtgatagagatc

SEQ ID NO: 108 - DNA, 33 bp, Homo sapiens
  mutation (13)..(15): substitutions
gcctcccagacgttgaagaaggtgatagagatc

SEQ ID NO: 109 - DNA, 33 bp, Homo sapiens
aggcaccatggctgggggcgcagcggattgcag

SEQ ID NO: 110 - DNA, 33 bp, Homo sapiens
  mutation (7)..(7): substitution
  mutation (20)..(22): substitutions
aggcacaatggctgggggcaccgcggattgcag

SEQ ID NO: 111 - DNA, 33 bp, Homo sapiens
agcttctgggaacggctgttggctcggcaatgt

SEQ ID NO: 112 - DNA, 33 bp, Homo sapiens
  mutation (27)..(27): substitution
  mutation (31)..(33): substitutions
agcttctgggaacggctgttggctcgccaattc

SEQ ID NO: 113 - DNA, 33 bp, Homo sapiens
ctctaccacgtgcgggaggatggctggatccga

SEQ ID NO: 114 - DNA, 33 bp, Homo sapiens
  mutation (6)..(6): substitution
  mutation (22)..(24): substitutions
ctctatcacgtgcgggaggatgactggatccga

(19) United States
(12) Patent Application Publication
(10) Pub. No.: US 2022/0186210 A1
(43) Pub. Date: Jun. 16, 2022

(54) METHOD FOR IDENTIFYING FUNCTIONAL ELEMENTS

(71) Applicants: Peking University, Beijing (CN); Edigene Inc., Beijing (CN)

(72) Inventors: Wensheng WEI, Beijing (CN); Yinan WANG, Beijing (CN); Yuexin ZHOU, Beijing (CN); Xinyi ZHANG, Beijing (CN); Di YUE, Beijing (CN); Ying LIU, Beijing (CN)

(73) Assignees: Peking University, Beijing (CN); Edigene Inc., Beijing (CN)

(21) Appl. No.: 17/593,811

(22) PCT Filed: Mar. 26, 2020
(86) PCT No.: PCT/CN2020/081283

§ 371 Date: Sep. 24, 2021

(30) Foreign Application Priority Data
Mar. 26, 2019 (CN) PCT/CN2019/079729

(51) Int. Cl. C12N 15/10; C12Q 1/6897; C12N 15/11

(52) U.S. Cl. CPC C12N 15/1079; C12Q 1/6897; C12N 2330/31; C12N 2310/20; C12N 2320/11; C12N 15/111

(57) ABSTRACT

Provided are a method for identifying functional elements of a genomic sequence and a library used for identifying functional elements of a genomic sequence.

### FIELD OF THE INVENTION

The present invention is related to a method for identifying functional elements of a genomic region or a protein of interest. Specifically, the invention is involved in a high-throughput strategy to identify elements critical for their functions in their native biological contexts.

### BACKGROUND OF THE INVENTION

RNA-guided CRISPR-associated protein 9 nucleases could introduce indels (insertions or deletions) and point mutations on targeted genomic loci through generating double strand breaks (DSBs) and consequently activating internal repair mechanisms, especially non-homologous end joining (NHEJ)^{(1, 2)}. Mutagenesis, especially that leading to reading frame-shift, could completely abolish gene expression, making CRISPR-Cas9 system a powerful tool for genome engineering^{(3, 4)}, and even for high-throughput functional screening⁽⁵⁻⁸⁾. To better understand the role of regulatory elements or protein-coding sequences with high resolution, CRISPR-mediated saturation mutagenesis has been employed with a relevant biological assay^{(9, 10)}. Because these attempts only collected indirect sequencing data from sgRNA-coding regions, their base-recognition resolution was limited. Moreover, it is unlikely to obtain complete functional domain or critical amino acid information using such strategy, especially if the protein of interest is dispensable for cell viability. Traditional methods are mainly in vitro biochemical assays, such as co-immunoprecipitation (Co-IP) combined with truncation mutagenesis⁽¹¹⁾, however, these techniques are time consuming, labor intensive and with low resolution, not to mention none of them are performed in native biological contexts. Hence a more accurate and comprehensive strategy and method is highly needed in the art for identifying functional elements for a protein or genomic sequence of interest.

### SUMMARY OF THE INVENTION

The present invention satisfies at least some of the aforementioned needs by providing a high-throughput strategy and method for identifying functional elements for a genomic region or a protein of interest, which is designated as CRESMAS (CRISPR-Empowered Saturation Mutagenesis combined with Assorted-DNA-fragment Sequencing). Specifically, the present invention applies saturation mutagenesis and retrieve only in-frame mutations (in-frame deletions and missense point mutations) that give rise to change of phenotype to identify critical sites related to functions of the genomic region or the protein, regardless of the essentiality of targeted genes.

Using this approach, the inventors mapped six proteins, three bacterial toxin receptors and three cancer drug targets, and acquired their comprehensive functional maps at single amino acid resolution, which contained both known domains or sites and novel amino acids critical for drug or toxin sensitivity. This novel method revealed comprehensive and precise single-amino-acid-substitution patterns on critical residues that would abolish protein function or confer drug resistance. The scalable CRESMAS strategy with profound accuracy and efficiency enables sequence-to-function mapping of variety of proteins at high resolution, and has the potential to accelerate mechanistic studies of protein function and drug resistance.

In one aspect, the present invention is related to a method for identifying functional elements for a protein of interest, comprising conducting saturation mutagenesis to provide multiplex mutations covering every amino acid by using CRISPR system, retrieving in-frame mutations that give rise to loss-of-function phenotypes, PCR amplifying sgRNA coding regions and cDNA of the target gene for sequencing analysis and building a computational pipeline to analyze the sequencing data to identify amino acids essential for the protein of interest. In one embodiment, the identification to the functional elements for the protein of interest is at single amino acid resolution. In one embodiment, the identification to the functional elements for the protein of interest is in its native biological context. In one embodiment, the in-frame mutations are in-frame deletions and missense point mutations.

In one embodiment, the saturation mutagenesis by using CRISPR system comprises designing sgRNAs for each amino acid spanning full length of the protein of interest. In one embodiment, each sgRNA is designed to affect about 10-bp (for example, 7-13, for example, 8-bp, 9-bp, 10-bp, 11-bp and 12-bp) around the DSB site. In one embodiment, the in-frame deletions comprise driver deletions as either “driver deletions” (containing only single amino acid deletions) or “passenger deletions” (containing multiple amino acid deletions).

In one embodiment, the computational pipeline comprises:

Mapping sequencing reads to the reference sequences of the target gene using public available bioinformatic tools, for example Bowtie2 2.3.2 and SAMtools 1.3.1.

Filtering the reads to retain those that carried only missense mutations or in-frame deletions,

For fragments containing missense mutations, computing the mutation ratio of each amino acid as follows:

$$mutation ratio = \frac{number of sequenced mutations of the amino acid}{total number of sequenced reads of the amino acid}$$

For fragments containing in-frame deletions, computing the deletion ratio of each amino acid as follows:

$$deletion ratio = \frac{number of sequenced deletions of the amino acid}{total number of sequenced reads of the amino acid}$$

Decoding the in-frame deletions and categorizing the in-frame deletions based on the number of amino acid deletions as either “driver deletions”, if they contain only single amino acid deletions, or “passenger deletions”, if they contain multiple amino acid deletions,

Computing the fold changes between the experimental and control groups,

Computing the essential score for each amino acid as follows:

for the mutation fold change, a null distribution is built based on all fold changes, and scoreₘᵤₜₐₜᵢₒₙ=−log10(P-value) was computed for each amino acid,

For the deletion fold change, a tunable parameter, α, is first applied to weight the driver deletion and passenger deletion as follows:

deletion fold change=driver fold change+α*passenger fold change, and then a null distribution is built via permutation 100 times, and score_{deletion}=−log10(P-value) is computed for each amino acid,

scoreₘᵤₜₐₜᵢₒₙ and score_{deletion} are normalized as follows:

$$s c o r {e}_{mutation} = \frac{( s c o r {e}_{mutation} - min ( s {core}_{mutation} ) )}{( max ( s {core}_{mutation} ) - min ( s {core}_{mutation} ) )} \\ s c o r {e}_{deletion} = \frac{( s {core}_{deletion} - min ( s {core}_{deletion} ) )}{( max ( {score}_{deletion} ) - min ( {score}_{deletion} ) )}$$

computing the weights of scoreₘᵤₜₐₜᵢₒₙ and score_{deletion} as follows:

$$a = number of amino acids with deletion fold change > 1$$

$$b = number of amino acids with mutation fold change > 1$$

$${w}_{mutation} = \frac{a}{a + b}$$

$${w}_{deletion} = \frac{b}{a + b}$$

computing the essential score as follows:

essential score=w_{GHIJIKLM}*score_{GHIJIKLM}+W_{STUTIKLM}*score_{STUTIKLM}.

In one embodiment, the method further comprises ranking the amino acids based on their functional importance according to the essential scores.

In one aspect, the present invention is related to a library used for CRESMAS to identify functional elements of genomic sequences comprising a plurality of CRISPR-Cas system guide RNAs comprising guide sequences that are capable of targeting a plurality of genomic sequences within at least one continuous genomic region, wherein the guide RNAs target at least 100 genomic sequences comprising non-overlapping cleavage sites upstream of a PAM sequence for every 1000 base pairs within the continuous genomic region.

In one embodiment, each guide RNA in the library is designed to affect about 10 bp (for example, 7-13, for example, 8-bp, 9-bp, 10-bp, 11-bp and 12-bp) around the DSB site. In one embodiment, the library comprises guide RNAs targeting genomic sequences upstream of every PAM sequence within the continuous genomic region. In one embodiment, the PAM sequence is specific to at least one Cas protein. In one embodiment, the CRISPR-Cas system guide RNAs are selected based upon more than one PAM sequence specific to at least one Cas protein. In one embodiment, the expression of the gene of interest is altered by said targeting by at least one guide RNA within the plurality of CRISPR-Cas system guide RNAs. In one embodiment, the library is introduced into a population of cells, preferably, a population of eukaryotic cells. In one embodiment, said targeting results in NHEJ of the continuous genomic region. In one embodiment, the targeting is of about 100 or more sequences, about 1,000 or more sequences, about 100,000 or more sequences.

In one embodiment, the targeting comprises introducing into each cell in the population of cells a vector system of one or more vectors comprising an engineered, non-naturally occurring CRISPR-Cas system comprising

I. a Cas protein or a polynucleotide sequence encoding a Cas protein, which is operably linked to a regulatory element, and

II. a CRISPR-Cas system guide RNA,

wherein components I and II are on the same or on different vectors, and wherein transcribed, the guide RNA comprising the guide sequence directs sequence-specific binding of a CRISPR-Cas system to a target sequence in the continuous genomic region, inducing cleavage of the continuous genomic region by the Cas protein.

In one embodiment, the one or more vectors are plasmid vectors. The regulatory element is an inducible promoter, preferably, the inducible promoter is a doxycycline inducible promoter.

In one aspect, the present invention is related to a CRESMAS method comprising:

(a) introducing the library of any of the preceding claims into a population of cells that are adapted to contain at least one Cas protein, wherein each cell of the population contains no more than one guide RNA;

(b) sorting the cells into at least two groups based on a change in cellular phenotype;

(c) determining relative representation of the guide RNAs present in each group, whereby genomic sites associated with the change in cellular phenotype are determined by the representation of guide RNAs present in each group;

(d) amplifying one or more cDNA or DNA sequences of the targeted one or more genes for sequencing;

(e) mapping the sequencing reads to reference sequences of the target genes;

(f) filtering the reads to retain those that carry only missense mutations or in-frame deletions; and

(g) determining the weight of each amino acid or nucleotide acid for the cellular phenotype by applying a bioinformatics pipeline.

In one embodiment, the change in cellular phenotype is increase or decrease of transcription and/or expression of a gene of interest. In one embodiment, the cells are sorted into a high expression group and a low expression group. In one embodiment, the change in cellular phenotype includes loss of function or gain of function. In one embodiment, the method is for identifying functional elements for a protein of interest at single amino acid resolution.

In one embodiment, the above method is for identifying a functional map of a noncoding RNA, promotor or enhancer. The only modification in protocol is to perform PCR amplification on the targeted region on the genome instead of cDNA in the situation of identifying functional elements of a protein of interest.

In one aspect, the present invention is related to a method of screening functional elements associated with resistance to a chemical compound comprising:

(a) introducing any of the library mentioned above into a population of cells that are adapted to contain a Cas protein, wherein each cell of the population contains no more than one guide RNA;

(b) treating the population of cells with the chemical compound; and

(c) determining the representation of guide RNAs after treatment with the chemical compound as compared to that before treatment, whereby genomic sites associated with resistance to the chemical compound are determined by enrichment of guide RNAs;

(d) amplifying one or more cDNA or DNA sequences of the targeted one or more genes for sequencing;

(e) mapping the sequencing reads to reference sequences of the target genes;

(f) filtering the reads to retain those that carry only missense mutations or in-frame deletions; and

(g) determining the weight of each amino acid or nucleotide acid for the resistance to the chemical compound by applying a bioinformatics pipeline.

In certain embodiments, the bioinformatics pipeline comprises:

(h) For fragments containing missense mutations, computing the mutation ratio of each amino acid as follows:

$$mutation ratio = \frac{number of sequenced mutations of the amino acid}{total number of sequenced reads of the amino acid}$$

(i) For fragments containing in-frame deletions, computing the deletion ratio of each amino acid as follows:

$$deletion ratio = \frac{number of sequenced deletions of the amino acid}{total number of sequenced reads of the amino acid}$$

(j) Decoding the in-frame deletions and categorizing the in-frame deletions based on the number of amino acid deletions as either “driver deletions”, if they contain only single amino acid deletions, or “passenger deletions”, if they contain multiple amino acid deletions,

(k) Computing the fold changes between the experimental and control groups,

(l) Computing the essential score for each amino acid as follows:
(1) for the mutation fold change, a null distribution is built based on all fold changes, and scoreₘᵤₜₐₜᵢₒₙ=−log10(P-value) is computed for each amino acid,

1(2) the deletion fold change, a tunable parameter, α, is first applied to weight the driver deletion and passenger deletion as follows:

deletion fold change=driver fold change+α*passenger fold change, and then a null distribution is built via permutation 100 times, and score_{deletion}=−log10(P-value) is computed for each amino acid,
(3) scoreₘᵤₜₐₜᵢₒₙ and score_{deletion} are normalized as follows:

$$s c o r {e}_{mutation} = \frac{( s c o r {e}_{mutation} - min ( s {core}_{mutation} ) )}{( max ( s {core}_{mutation} ) - min ( s {core}_{mutation} ) )} \\ s c o r {e}_{deletion} = \frac{( s {core}_{deletion} - min ( s {core}_{deletion} ) )}{( max ( {score}_{deletion} ) - min ( {score}_{deletion} ) )}$$

(4) computing the weights of scoreₘᵤₜₐₜᵢₒₙ and score_{deletion} as follows:

$$a = number of amino acids with deletion fold change > 1$$

$$b = number of amino acids with mutation fold change > 1$$

$${w}_{mutation} = \frac{a}{a + b}$$

$${w}_{deletion} = \frac{b}{a + b}$$

(5) computing the essential score as follows:

essential score=w_{GHIJIKLM}*score_{GHIJIKLM}+w_{STUTIKLM}*score_{STUTIKLM}.

In the method herein, the chemical compound can be any chemical compound affecting the structure and/or function of one or more genomic regions or proteins in a eukaryotic cell. For example, it can be a toxin or drug, as exemplified herein. In some embodiments, the eukaryotic cell is a human cell.

In one aspect, the present invention is related to a method for identifying functional elements for a protein of interest, comprising conducting saturation mutagenesis to the protein of interest by disrupting the genomic gene coding for the protein by using CRISPR-Cas system introduced into a population of cells, determining disrupted genomic sites associated with change of phenotype by DNA sequencing, sequencing the cDNA of the target gene, retrieving in-frame mutations that give rise to the change of phenotype, and building a bioinformatics pipeline to analyze the sequencing data to identify functional elements of the protein of interest at single amino acid resolution. In this method, the identification of the functional elements for the protein of interest is in its native biological context.

In the method, the in-frame mutations are in-frame deletions and missense point mutations. In certain embodiments, the disrupting comprises introducing into each cell in the population of cells a vector system of one or more vectors comprising an engineered, non-naturally occurring CRISPR-Cas system comprising

I. a Cas protein or a polynucleotide sequence encoding a Cas protein, which is operably linked to a regulatory element, and

II. a guide RNA targeting the genomic gene coding for the protein,

wherein components I and II are on the same or on different vectors, and wherein transcribed, the guide RNA comprising the guide sequence directs sequence-specific binding of a CRISPR-Cas system to a target sequence in the genomic gene, inducing cleavage of the genomic region by the Cas protein.

In one embodiment, the one or more vectors are plasmid vectors. In one embodiment, the regulatory element is an inducible promoter. In one embodiment, the guide RNAs target at least 100 genomic sequences comprising non-overlapping cleavage sites upstream of a PAM sequence for every 1000 base pairs within the genomic gene. In one embodiment, each guide RNA is designed to affect about 10 bp (for example, 7-13 bp, for example, 8 bp, 9 bp, 10 bp, 11 bp, 12 bp) around the DSB site. In one embodiment, the library comprises guide RNAs targeting genomic sequences upstream of every PAM sequence within the genomic gene. In one embodiment, the PAM sequence is specific to at least one Cas protein. In one embodiment, the CRISPR-Cas system guide RNAs are selected based upon more than one PAM sequence specific to at least one Cas protein. In one embodiment, the expression of the gene of interest is altered by said targeting by at least one guide RNA within the plurality of CRISPR-Cas system guide RNAs. In one embodiment, said targeting results in NHEJ of the genomic gene.

In one aspect, the present invention is related to a method for modifying a gene or protein by mutating the functional elements, for example the genomic sites or amino acid sites which are identified by any method of the invention as critical for the function of the genomic gene of protein. Also contemplated are variant proteins with amino acid substitutions and/or deletions at the amino acid sites identified by the method as critical for the function of proteins.

### BRIEF DESCRIPTION OF THE DRAWING

FIGS. 1A-1B. CRESMAS workflow. Library screening is conducted by drug or toxin treatment, followed by the amplification of sgRNA barcodes and targeted gene's cDNA for NGS. The reads carrying only missense mutations are collected for point mutation fold change calculation and mutation pattern analysis. Reads containing in-frame deletions are categorized by the number of amino acid (a.a.) in deletions and gathered to compute deletion fold change. The essential scores are calculated by leveraging both information from in-frame deletions and mis sense mutations.

FIGS. 2A-2E. Experimental conditions for CRESMAS screening. FIG. 2A Dosage effects of three cancer drugs on HeLa cell death for the indicated treatment times. FIG. 2B Coverage of sgRNAs for each gene in the screens, with the assumption that each sgRNA affects the 10 bp upstream and downstream from its cutting site. The x-axis indicates the number of sgRNAs covered for each amino acid. The y-axis indicates the number of amino acids (a.a.) affected by the sgRNAs. FIG. 2C Distribution of sgRNA sequences in the control libraries. FIG. 2D Schematic representation of the PCR amplification of target cDNAs. The primers employed for the different genes are listed in Table 1. FIG. 2E PCR amplification of target cDNAs (left) and shearing of DNA fragments to an average length of 250 bp (right).

FIGS. 3A-3B. Library quality and editing-type distribution. FIG. 3A Percentages of point mutations, insertions and deletions detected for each gene in the control group and two replicates after screening. FIG. 3B Scatter plot of sgRNA fold changes after screening on a log scale between two replicates.

FIGS. 4A-4B. Scatter plot of the deletion fold changes and point mutation fold changes of the replicates. FIG. 4A Scatter plot of deletion fold changes after screening between two replicates. FIG. 4B Scatter plot of point mutation fold changes after screening between two replicates.

FIGS. 5A-5C. CRESMAS identification of critical amino acids that are essential for ANTXR1 in mediating PA toxicity. FIG. 5A Evaluation of sgRNAs targeting ANTXR1 in PA screening. The location of each sgRNA relative to the ANTXR1 protein is indicated along the x-axis. FIG. 5B Deletion and point mutation fold changes corresponding to each amino acid. A multi-domain schematic diagram of ANTXR1 is presented under the plot, with the PA binding site indicated. FIG. 5C Essential score of each amino acid of ANTXR1. Top-ranked hits are shown in dark gray, among which, known critical amino acids are shown in triangle.

FIGS. 6A-6C. CRESMAS identification of critical amino acids that are essential for CSPG4 in mediating TcdB toxicity. FIG. 6A Evaluation of sgRNAs targeting CSPG4 in TcdB screening. The location of each sgRNA relative to the CSPG4 protein is indicated along the x-axis. FIG. 6B Deletion and point mutation fold changes corresponding to each amino acid. A multi-domain schematic diagram of CSPG4 is presented under the plot, with the TcdB binding site indicated. FIG. 6C Essential score of each amino acid of CSPG4. Top-ranked hits are shown in dark gray.

FIGS. 7A-7D CRESMAS identification of critical amino acids essential for HBEGF in mediating DT toxicity. FIG. 7A Evaluation of sgRNAs targeting HBEGF in DT screening. The location of each sgRNA relative to the HBEGF protein is indicated along the x axis. The location of sgRNA is defined as the sgRNA's cutting site and the fold change is the average fold change of sgRNAs targeting the codon of each amino acid. FIG. 7B Deletion and point mutation fold change corresponding to each amino acid. Grey bars represent multiple amino acid deletions. The width of grey bar correlates the number of amino acids that were deleted together. The grey scale for each single amino acid was assigned to 10%. The grey scale was overlaid to indicate the statistic importance of any particular amino acid in diverse deletion patterns. The asterisk indicates known residue critical for protein function. A multi-domain schematic diagram of HBEGF is presented under the plot, with EGF-like domain indicated, a known binding region for DT. FIG. 7C The essential score of each amino acid of HBEGF. Top ranked hits are in dark grey, and known critical amino acids are in triangle. FIG. 7D Effect of single-amino-acid deletion on cell susceptibility to DT. Cells were treated with different concentrations of DT, and the MTT cytotoxicity assay was performed 48 hour after toxin treatment. Data are presented as the mean±s.d., n=5.

FIGS. 8A-8C CRESMAS identification of critical amino acids that are essential for HPRT1 in 6-TG killing. FIG. 8A Evaluation of sgRNAs targeting HPRT1 in the bortezomib screen. The location of each sgRNA relative to the HPRT1 protein is indicated along the x-axis. FIG. 8B Deletion and point mutation fold changes corresponding to each amino acid. A multi-domain schematic diagram of HPRT1 is presented under the plot. FIG. 8C Essential score of each amino acid of HPRT1. Top-ranked hits are shown in dark gray.

FIGS. 9A-9E CRESMAS identification of critical amino acids essential for PSMBS to Bortezomib killing. FIG. 9A Evaluation of sgRNAs targeting PSMBS in Bortezomib screening. The location of each sgRNA relative to the PSMBS protein is indicated along the x axis. FIG. 9B Deletion and point mutation fold change corresponding to each amino acid. FIG. 9C The essential score of each amino acid of PSMBS. Top ranked hits are in dark grey, and known critical amino acids are in triangle. FIG. 9D MTT viability assay for the effects of indicated point mutations of PSMBS on cell susceptibility to Bortezomib. FIG. 9E Effects of indicated point mutations of PSMBS on cell susceptibility to Bortezomib. Data are presented as the mean±s.d., n=6.

FIGS. 10A-10D CRESMAS identification of critical amino acids that are essential for PLK1 in BI2536 killing. FIG. 10A Evaluation of sgRNAs targeting PLK1 in the bortezomib screen. The location of each sgRNA relative to the PLK1 protein is indicated along the x-axis. FIG. 10B Deletion and point mutation fold changes corresponding to each amino acid. FIG. 10C Essential score of each amino acid of PLK1. Top-ranked hits are shown in dark gray, and known critical amino acids are shown in triangle. FIG. 10D MTT viability assay for determining the effects of the indicated point mutations in PLK1 on the susceptibility of cells to BI2536.

FIG. 11 Sequencing chromatogram of amino acid mutations in PSMBS from pooled cells with or without ssODN donor transfection. The mutated amino acids are shown.

FIG. 12 Sequence information for bortezomib-resistant cell clones. sgRNA sequences are underlined; nucleotides with shadowing represent the PAM sequence; letters with dots underneath and letters boxed indicate wild-type and mutated amino acids, respectively.

FIGS. 13A-13H Point mutation pattern of top ranked hits of PSMB5 and PLK1. Heat maps show the point mutation diversity of a specific amino acid among the top ranked hits of PSMB5 FIG. 13A and PLK1 FIG. 13B. Bar charts indicate the percentage of 20 amino acid substitutions for V90PSMB5 FIG. 13C, A386PLK1 FIG. 13D, M104PSMB5 and C122PSMB5 FIG. 13E, F183PLK1 and R136PLK1 FIG. 13F, A105PSMB5 and A43PSMB5 FIG. 13G 20 amino acids are classified into 4 groups (nonpolar, polar, acidic and basic) shown as different bar forms according to their properties of side chains. The original amino acids are highlighted in grey shadow. FIG. 13H Scatter plot of amino acid distribution between A105PSMB5 and A43PSMB5.

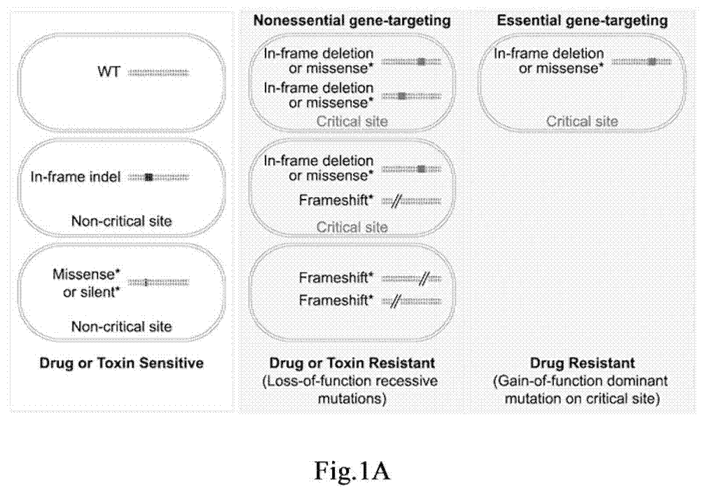

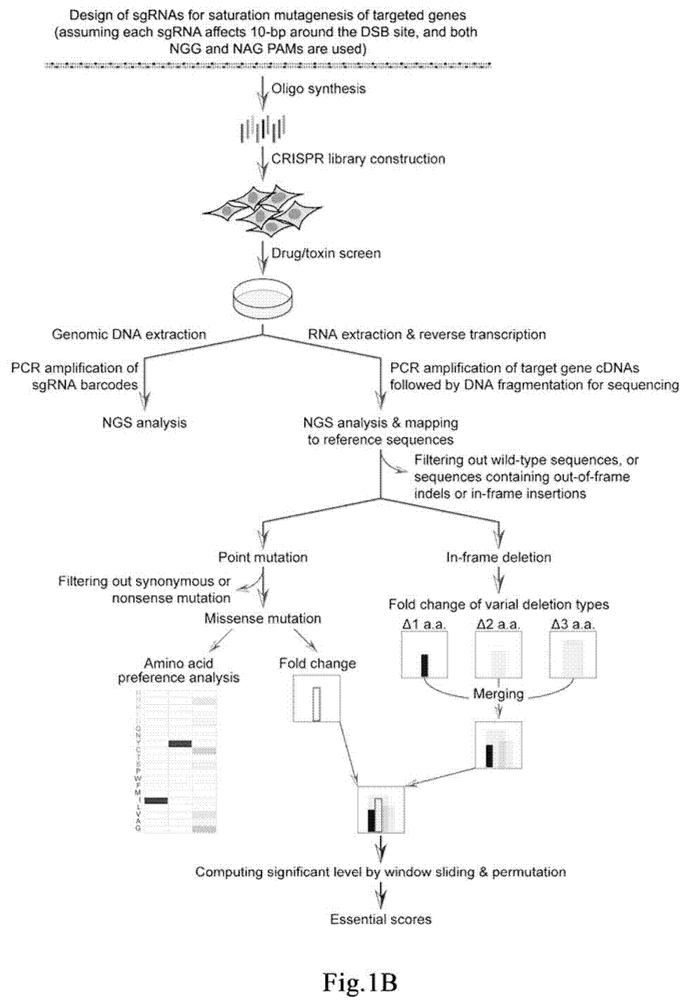

### DETAILED DESCRIPTION OF THE INVENTION

The methods and tools described herein relate to systematically interrogating genomic regions in order to allow the identification of relevant functional units which can be of interest for genome editing. Accordingly, in one aspect the invention provides methods for interrogating a genomic region said method comprising generating a deep scanning mutagenesis library and interrogating the phenotypic changes within a population of cells modified by introduction of said library.

One aspect of the invention thus comprises a deep scanning mutagenesis library that may comprise a plurality of CRISPR-Cas system guide RNAs that may comprise guide sequences that are capable of targeting genomic sequences within at least one continuous genomic region. More particularly it is envisaged that the guide RNAs of the library should target a representative number of genomic sequences within the genomic region. For example, the guide RNAs should target at least 50, more particularly at least 100, genomic sequences within the envisaged genomic region.

The ability to target a genomic region is determined by the presence of a PAM (protospacer adjacent motif); that is, a short sequence recognized by the CRISPR complex. The precise sequence and length requirements for the PAM will differ depending on the CRISPR enzyme which will be used, but PAMs are typically 2-5 base pair sequences adjacent the protospacer (that is, the target sequence). PAM sequences known in the art, and the skilled person will be able to identify PAM sequences for use with a given CRISPR enzyme. In particular embodiments, the PAM sequence can be selected to be specific to at least one Cas protein. In alternative embodiments, the guide sequence RNAs can be selected based upon more than one PAM sequence specific to at least one Cas protein.

In particular embodiments, the library contains at least 100 genomic sequences comprising non-overlapping cleavage sites upstream of a PAM sequence for every 1000 base pairs within the genomic region. In particular embodiments the library comprises guide RNAs targeting genomic sequences upstream of every PAM sequence within the continuous genomic region.

This library comprises guide RNAs that target a genomic region of interest of an organism. In some embodiments of the invention the organism or subject is a eukaryote (including mammal, including human) or a non-human eukaryote or a non-human animal or a non-human mammal. In some embodiments, the organism or subject is a non-human animal, and may be an arthropod, for example, an insect, or may be a nematode. In some methods of the invention the organism or subject is a plant. In some methods of the invention the organism or subject is a mammal, for example, a human or non-human mammal. A non-human mammal may be for example a rodent (preferably a mouse or a rat), an ungulate, or a primate. In some methods of the invention the organism or subject is algae, including microalgae, or is a fungus.

The methods and tools provided herein are particularly advantageous for interrogating a continuous genomic region. Such a continuous genomic region may comprise up to the entire genome, but particularly advantageous are methods wherein a functional element of the genome is interrogated, which typically encompasses a limited region of the genome, such as a region of 50-100 kb of genomic DNA. Of particular interest is the use of the methods for the interrogation of coding genomic regions. A skilled person in the art can understand that the methods of the present invention can also be used for interrogation of non-coding genomic regions, such as regions 5′ and 3′ of the coding region of a gene of interest by modification in protocol to perform PCR amplification on the targeted region on the genome instead of cDNA in the scenario of interrogation of a protein of interest.

The CRISPR/Cas system can be used in the present invention to specifically target a multitude of sequences within a continuous genomic region of interest. The targeting typically comprises introducing into each cell of a population of cells a vector system of one or more vectors comprising an engineered, non-naturally occurring CRISPR-Cas system comprising: at least one Cas protein and guide RNA. In these methods, the Cas protein and the guide RNA may be on the same or on different vectors of the system and are integrated into each cell, whereby each guide sequence targets a sequence within the continuous genomic region in each cell in the population of cells. The Cas protein is operably linked to a regulatory element to ensure expression in said cell, more particularly a promoter suitable for expression in the cell of the cell population. In particular embodiments, the promoter is an inducible promoter, such as a doxycycline inducible promoter. When transcribed within the cells of the cell population, the guide RNA comprising the guide sequence directs sequence-specific binding of a CRISPR-Cas system to a target sequence in the continuous genomic region. Typically binding of the CRISPR-Cas system induces cleavage of the continuous genomic region by the Cas protein.

The application provides methods of screening for functional elements associated with a change in a phenotype. The change in phenotype can be detectable at one or more levels including at DNA, RNA, protein and/or functional level of the cell. The change in phenotype can be detectable in cellular survival, growth, immune reaction, resistance to a chemical compound, such as a toxin or drug.

The methods of screening for genomic sites associated with a change in phenotype comprise introducing the library of guide RNAs targeting the genomic region of interest as envisaged herein into a population of cells. Typically the cells are adapted to contain a Cas protein. However, in particular embodiments, the Cas protein may also be introduced simultaneously with the guide RNA. The introduction of the library into the cell population in the methods envisage herein is such that each cell of the population contains no more than one guide RNA. Hereafter, the cells are typically sorted based on the observed phenotype and the genomic sites associated with a change in phenotype are identified based on whether or not they give rise to a change in phenotype in the cells. Typically, the methods involve sorting the cells into at least two groups based on the phenotype and determining relative representation of the guide RNAs present in each group, and genomic sites associated with the change in phenotype are determined by the representation of guide RNAs present in each group.

The application similarly provides methods of screening for genomic sites associated with resistance to a chemical compound whereby the cells are contacted with the chemical compound and screened based on the phenotypic reaction to said compound. More particularly such methods may comprise introducing the library of CRISPR/Cas system guide RNAs envisaged herein into a population of cells (that are either adapted to contain a Cas protein or whereby the Cas protein is simultaneously introduced), treating the population of cells with the chemical compound; and determining the representation of guide RNAs after treatment with the chemical compound at a later time point as compared to an early time point. In these methods the genomic sites associated with resistance to the chemical compound are determined by enrichment of guide RNAs.

In particular embodiments, the methods may further comprise sequencing the region comprising the genomic site or by whole genome sequencing.

The application further relates to methods for screening for functional elements related to drug resistance using the methods of the present invention.

Further embodiments described herein relate to therapeutic methods and tools involving genomic disruption of one or more functional regions of a gene identified by the methods herein disclosed. These and Further embodiments described herein are based in part to the discovery of functional regions in a genomic region or a protein of interest.

In specific methods exemplified in the present application, to maximize the coverage density, both types of protospacer-adjacent motifs (PAMs), NGG and NAG, are encompassed for the design of sgRNAs. After library screening using cancer drugs or toxins, the genomic DNA was extracted for conventional PCR amplification of sgRNA barcodes followed by NGS analysis. Meanwhile, PCR amplification of targeted genes from reverse transcription of RNAs were conducted and the fragmented PCR products around 250-bp in length were subjected to NGS. We then filtered out wild-type sequences or those containing out-of-frame indels or in-frame insertions so that only those sequences containing either point mutation or in-frame deletion were retained for further analysis. For point mutation, we went on filtering out synonymous or nonsense mutation and kept only those containing missense mutation. In case of in-frame deletion, we categorized mutation types by the number of amino acid deletion they caused for each read, and then classified them as either “driver deletions” if they contained only single-amino-acid deletions or “passenger deletions” if they contained multiple-amino-acid deletions. After decoding deletion patterns, the deletion fold changes were computed. Similarly, the fold changes for missense mutations were also calculated. Next, we leveraged all information from filtered reads by applying a window sliding on the target gene to compute weighted average of fold changes for missense mutation, driver deletion and passenger deletion. We then inferred the significant level of the weighted average by permutation and acquired the essential score for each amino acid. The score counted both the in-frame deletion and point mutation scenarios and quantified the essentiality of each amino acid so that we could rank the amino acids based on their functional importance. Meanwhile, we attempted to obtain the amino acid substitution pattern by counting the percentage of missense mutations for each amino acid. This streamlined workflow and a bioinformatics pipeline were designed to enable us to identify critical functional elements of proteins in their native biological contexts.

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PGR 2: A PRACTICAL APPROACH (M.J. MacPherson, B.D. Hames and GR. Taylor eds. (1995)), Harlow and Lane, eds. (1988) ANTIBODIES, A LABORATORY MANUAL, and ANIMAL CELL CULTURE (R. L Freshney, ed. (1987)).

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 2nd ed., Cold Spring Harbor Press, Plainsview, New York (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

In genetics, a “nonsense mutation” is a point mutation in a sequence of DNA that results in a premature stop codon, or a nonsense codon in the transcribed mRNA, and in a truncated, incomplete, and usually nonfunctional protein product. The functional effect of a nonsense mutation depends on the location of the stop codon within the coding DNA. For example, the effect of a nonsense mutation depends on the proximity of the nonsense mutation to the original stop codon, and the degree to which functional subdomains of the protein are affected. A nonsense mutation differs from a “missense mutation”, which is a point mutation where a single nucleotide is changed to cause substitution of a different amino acid.

A “synonymous substitution or mutation” is the evolutionary substitution of one base for another in an exon of a gene coding for a protein, such that the produced amino acid sequence is not modified. This is possible because the genetic code is “degenerate”, meaning that some amino acids are coded for by more than one three-base-pair codon; since some of the codons for a given amino acid differ by just one base pair from others coding for the same amino acid, a mutation that replaces the “normal” base by one of the alternatives will result in incorporation of the same amino acid into the growing polypeptide chain when the gene is translated.

A protein contains both dispensable and indispensable regions, mutations on latter parts would abolish its function. On its corresponding DNA-coding sequences, any mutation leading to reading frame shift has high chance of disrupting gene expression hence its function, no matter whether the mutation occurs in the critical or non-critical site. In cases of protein targets of cancer drugs or bacterial toxins, in-frame deletion or point mutation (except for nonsense mutation) does not produce resistance phenotype when such mutation hits the non-critical site. For non-essential gene, disruption of every allele is a necessity to achieve “loss-of-function phenotype”. These recessive mutation types could be one of the following: frameshift indel, in-frame deletion or missense point mutation affecting critical site. For essential gene, the only drug-resistance scenario is either in-frame deletion or missense mutation affecting the critical site for drug targeting without altering protein's expression and thus its essential role for cell viability. These mutations are dominant and thus a proper mutation in one allele is sufficient to achieve “gain-of-function phenotype”.

In a wild-type diploid cell, there are two wild-type alleles of a gene, both making normal gene product. In heterozygotes (the crucial genotypes for testing dominance or recessiveness), the single wild-type allele may be able to provide enough normal gene product to produce a wild-type phenotype. In such cases, “loss-of-function mutations” are recessive. In some cases, the cell is able to “upregulate” the level of activity of the single wild-type allele so that in the heterozygote the total amount of wild-type gene product is more than half that found in the homozygous wild type. However, mutation events confer some new function on the gene. In a heterozygote, the new function will be expressed, and therefore the “gain-of-function mutation” most likely will act like a dominant allele and produce some kind of new phenotype.

“Saturation mutagenesis” is a random mutagenesis technique, in which each single codon or set of codons is randomized to produce all possible amino acids at the position.

A “codon” is a set of three nucleotides, a triplet that code for a certain amino acid. The first codon establishes the reading frame, whereby a new codon begins. A protein's amino acid backbone sequence is defined by contiguous triplets. Codons are key to translation of genetic information for the synthesis of proteins. The “reading frame” is set when translating the mRNA begins and is maintained as it reads one triplet to the next. The reading of the genetic code is subject to three rules the monitor codons in mRNA. First, codons are read in a 5′ to 3′ direction. Second, codons are nonoverlapping and the message has no gaps. The last rule, as stated above, that the message is translated in a fixed “reading frame”.

A “frameshift mutation”, also called a framing error or a reading frame shift, is a genetic mutation caused by indels (insertions or deletions) of a number of nucleotides in a DNA sequence that is not divisible by three. Due to the triplet nature of gene expression by codons, the insertion or deletion can change the reading frame, resulting in a completely different translation from the original. A frameshift mutation will in general cause the reading of the codons after the mutation to code for different amino acids. The frameshift mutation will also alter the first stop codon (“UAA”, “UGA” or “UAG”) encountered in the sequence. The polypeptide being created could be abnormally short or abnormally long, and will most likely not be functional.

“Out-of-frame indels” mean the insertions and/or deletions (indels) which cause the reading of the genetic code out of “reading frame”, while “in-frame deletion” means the deletion of a number of nucleotides in a DNA sequence that is divisible by three, and thus the deletion does not change the reading frame.

“CRISPR system” herein refers collectively to transcripts and other elements involved in the expression of or directing the activity of CRISPR-associated (“Cas”) genes, including sequences encoding a Cas gene, a tracr (trans -activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In some embodiments, one or more elements of a CRISPR system is derived from a type I, type II, or type III CRISPR system.

Within an expression vector, “operably linked” is intended to mean that the nucleotide sequence of interest is linked to the regulatory sequence(s) in a manner which allows for expression of the nucleotide sequence (e.g., in an in vitro transcription/translation system or in a target cell when the vector is introduced into the target cell).

In the context of formation of a CRISPR complex, “target sequence” refers to a sequence to which a guide sequence is designed to have complementarity, where hybridization between a target sequence and a guide sequence promotes the formation of a CRISPR complex. Full complementarity is not necessarily required, provided there is sufficient complementarity to cause hybridization and promote formation of a CRISPR complex.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part, of a CRISPR complex, such as by hybridization along at least a portion of the tracr sequence to all or a portion of a tracr mate sequence that is operably linked to the guide sequence.

In some embodiments, the tracr sequence has sufficient complementarity to a tracr mate sequence to hybridize and participate in formation of a CRISPR complex. As with the target sequence, it is believed that complete complementarity is not needed, provided there is sufficient to be functional. In some embodiments, the tracr sequence has at least 50%, 60%, 70%, 80%, 90%, 95% or 99% of sequence complementarity along the length of the tracr mate sequence when optimally aligned.

In some embodiments, one or more vectors driving expression of one or more elements of a CRISPR system are introduced into a host cell such that expression of the elements of the CRISPR system direct formation of a CRISPR complex at one or more target sites. In another embodiment, the host cell is engineered to stably express Cas9 and/or OCT1.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 70%, 75%, 80%, 85%, 90%, 91%, 92%, 93%, 94%, 95%, 96%, 97%, 98%, 99% or more. Optimal alignment may be determined with the use of any suitable algorithm for aligning sequences, non-limiting example of which include the Smith-Waterman algorithm, the Needleman-Wimsch algorithm, algorithms based on the Burrows-Wheeler Transform (e.g. the Burrows Wheeler Aligner), ClustalW, Clustai X, BLAT, Novoalign (Novocraft Technologies, ELAND (I!fumma, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, 11, 10 or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

In some embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, historic modification activity, RNA cleavage activity and nucleic acid binding activity.

In some aspects, the invention provides methods comprising delivering one or more polynucleotides, such as or one or more vectors as described herein, one or more transcripts thereof, and/or one or proteins transcribed therefrom, to a host cell. The invention serves as a basic platform for enabling targeted modification of DNA -based genomes. It can interface with many delivery systems, including but not limited to viral, liposome, electroporation, microinjection and conjugation. In some aspects, the invention further provides cells produced by such methods, and organisms (such as animals, plants, or fungi) comprising or produced from such cells. In some embodiments, a CRISPR enzyme in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes for delivery to the cell.

CRISPR/Cas9 is used in the present invention for screening experiments, due to the relative ease of designing gRNAs and the ability of Cas9 to modify virtually any genetic locus. In the screening experiments, CRISPR pooled libraries or CRISPR libraries consist of thousands of plasmids, each containing a gRNA toward a different target sequence spanning the full length of the protein of the interest. Specifically, to achieve saturation mutagenesis on the protein of interest, the sgRNAs are designed to encompass both types of protospacer-adjacent motifs (PAMs), NGG and NAG, and each sgRNA is designed to affect 10-bp around the DSB site for maximizing the coverage density. The CRISPR screening experiment can be forward genetic screening, where the desired phenotype is known, but the critical amino acids of the protein are not. Typically, CRISPR-based screens are carried out by using lentivirus to deliver a “pooled” gRNA library to a mammalian Cas9 expressing cell line. Following transduction with the gRNA library, mutant cells are screened for a phenotype of interest (e.g., survival, drug or toxin resistance, growth or proliferation) to identify amino acids critical for the function of the protein and the desired phenotype.

The pooled lentiviral gRNA library is a heterogeneous mixture of lentiviral transfer vectors with each vector encoding an individual gRNA for a specific sequence and with several gRNAs targeting each sequence present in the library.

Performing a screen using a pooled lentiviral CRISPR library is a multi-step processes including library amplification, cellular transduction, genetic screening and data analysis. In brief, the initial stock of gRNA-containing plasmids are “amplified” to increase the total amount of DNA, and the amplified library is then used to generate lentivirus containing either the gRNA alone or gRNA +Cas9. For single-vector libraries, mutant cells are generated in one step by transducing wild-type cells with lentivirus containing both a single gRNA and Cas9. In most cases, for multi-vector libraries, cells expressing Cas9 are transduced with the gRNA library. In both cases, transduced cells are selected to enrich those containing both gRNA and Cas9 and the resulting population of mutant cells are screened for the particular phenotype of interest. Next-generation sequencing (NGS) is carried out on genomic DNA from the final population to identify gRNAs that are enriched or depleted during screening. Lastly, a bioinformatic pipeline is designed to analyze the retrieved data.

Library Amplification

Pooled lentiviral CRISPR gRNA libraries are often delivered as a DNA aliquot and in most cases the quantity of DNA is insufficient to be used in an experiment. In such cases, the first step is to “amplify” the library, meaning to increase the amount of plasmid DNA while maintaining the relative proportion of each individual gRNA plasmid within the total population. Amplification is carried out by transforming the library DNA into bacteria and harvesting the plasmid DNA after a period of bacterial growth. For most libraries, electroporation is used rather than chemical transformation due to the increased transformation efficiency using electroporation. In most cases, transformed bacteria are grown on LB agar plates containing the appropriate antibiotic, as growth on plates helps maintain library representation and reduces the probability that fast-growing plasmids will become enriched during amplification. An estimation of the number of gRNA plasmids that were transformed and amplified can be obtained by performing a dilution plating assay. To do this, a sample of the transformation is diluted and plated onto LB plates containing antibiotic and the number of colonies that grow on the plates is used as an indirect measure of the total number of gRNA plasmids present in the amplified library. This analysis serves as an important control to know what is in the final amplified library before it is used in a functional screen.

Cellular Transduction

Once the library has been amplified and the representation confirmed, the next step is to generate lentivirus containing the pooled gRNA library. Generally, HEK293T cells are transfected with the CRISPR library and appropriate packaging and envelope vectors (e.g., psPAX2; Addgene, plasmid #12260 from Didier Trono's lab, pMD2.G; Addgene, plasmid #12259 from Didier Trono's lab, pVSVG and pR8.74 from Addgene). Alternatively, a lentiviral packaging cell type can be transfected with the gRNA library alone. Most protocols recommend collecting the medium >48 hours after transfection, but some optimization may be required as maximal viral titer will vary depending on the specific library in question.

The goal of the transduction step is to generate a population of mutant cells that stably co-expresses Cas9 and a single gRNA. Single-vector libraries containing both gRNA and Cas9 are easier to use than multi-vector systems since mutant cells can be generated directly from wild-type cells in a single step. Afterwards, selection is carried out after lentiviral transduction to isolate a population of cells positive for Cas9 and a gRNA. If antibiotic selection is used, a kill curve should be performed to determine the optimum antibiotic concentration to select only those cells that contain Cas9 and gRNA.

In theory, any cell type can be used for screening, but the final population of cells must be in sufficient quantity to maintain library representation prior to screening. The exact number of cells required for a screen will vary based on the specific library in question. The easiest way to understand this is to work backwards from the final, mutant cell population and determine the exact number of cells required at the beginning of a screen. Take, for example, a hypothetical library of 10,000 gRNAs that is to be used at 100× representation. The bare minimum of cells required to conduct a screen using this library would be 10,000 gRNAs×100 cells/gRNA=10⁶ cells (not including control conditions for screening). Each cell in the final population must contain only one gRNA, as delivery of multiple gRNAs to a single cell could result in multiple genetic alterations, making it unclear which mutation actually leads to the observed phenotype. Thus, most protocols recommend transducing cells with the lentiviral gRNA library at a multiplicity of infection (MOI) of <1 (i.e., less than one viral particle per cell).

Genetic Screening

Genetic screens can be broadly defined as either positive, which reveal gRNAs that are enriched during screening, or negative, which reveal gRNAs that are depleted during screening. CRISPR libraries can be used in positive selection drug screens to search for genes that, when mutated, confer resistance to chemotherapeutic drugs. In positive-selection drug screens, it may be important to determine the optimum concentration to kill all wild-type cells (kill-curve), such that treating a population of mutant cells selectively enriches cells whose genetic modification promotes drug resistance. Furthermore, it is essential to compare the final gRNA counts within the genomic DNA to a control condition (such as a vehicle control) that is run in parallel, to control for drug-independent changes in gRNA distribution, such as the effect of a given gRNA on cell growth in the absence of drug or effects of the vehicle itself. Negative screens, on the other hand, seek to identify gRNAs that drop out of the population during screening, indicating that they are at a selective disadvantage relative to the rest of the population. A straightforward example of a negative selection screen is to allow mutant cells to grow for a defined period of time, and then compare the gRNA distribution at a later time point to an initial time point.

Data Analysis

The end result of any successful screen is to obtain a population of mutant cells that are either enriched (positive selection) or depleted (negative selection) in gRNAs whose target sequences or elements are essential for the observed phenotype. Therefore, the goal of the data analysis step is to identify the gRNAs and sequences or elements that have been depleted or enriched in the experimental group. Since the end population of cells could conceivably contain thousands of different gRNAs, analysis of the genomic sequence requires the use of next-generation sequencing (NGS). Each individual gRNA plasmid contains a barcode that differentiates that gRNA from all others present in the genomic DNA. Thus, the first step in analyzing data from a CRISPR screen is to amplify the gRNA relative to the genomic DNA using PCR and perform NGS to identify which gRNAs are present in the final mutant cell population. The end result of NGS is a raw count of all barcodes, from which the gRNA sequence and target gene can be deduced.

One way to determine whether a sequence or element is a “hit” is by qualitatively comparing how many gRNAs targeting that sequence or element are enriched, or depleted, within a given sample. As pointed out in earlier sections, libraries typically contain multiple different gRNAs per gene and consistent enrichment or depletion across multiple gRNAs for a specific gene is strong evidence that a particular sequence is important for the observed phenotype. Having several gRNAs also serves as an internal control for off-target effects, since it is unlikely that two different gRNAs toward the same target will have the same off-target effect. However, setting arbitrary thresholds to define hits (e.g., two out of six gRNAs qualifies as a “hit”) can be a potential source of bias or lead to false positive or negative results. To circumvent this, various statistical analyses can also be used to determine hits in an unbiased manner. Since each screen will be different, it is important to understand which statistical approach is best suited for a particular screen.

In the process of data analysis of the present invention, those data are to be filtered out with respect of wild-type sequences or sequences containing out-of-frame indels or in-frame insertions so that only sequences containing either point mutation or in-frame deletion are retained for further analysis. For point mutation, filtering out synonymous or nonsense mutation and kept only those containing missense mutation. For in-frame deletion, mutations need to be categorized by the number of amino acid deletion they caused for each read as either driver deletions if they contained only single-amino-acid deletions or passenger deletions if they contained multiple-amino-acid deletions. The bioinformatical analysis specifically comprises:

computing the mutation ratio of each amino acid as follows for fragments containing mis sense mutations:

$$mutation ratio = \frac{number of sequenced mutations of the amino acid}{total number of sequenced reads of the amino acid}$$

computing the deletion ratio of each amino acid as follows for fragments containing in-frame deletions:

$$deletion ratio = \frac{number of sequenced deletions of the amino acid}{total number of sequenced reads of the amino acid}$$

Computing the essential score for each amino acid as follows:

for the mutation fold change, a null distribution is built based on all fold changes, and scoreₘᵤₜₐₜᵢₒₙ=−log10(P-value) was computed for each amino acid,

For the deletion fold change, a tunable parameter, α, is first applied to weight the driver deletion and passenger deletion as follows:

deletion fold change=driver fold change+α*passenger fold change, and then a null distribution is built via permutation 100 times, and score_{deletion}=−log10(P-value) is computed for each amino acid,

scoreₘᵤₜₐₜⱼₒₙ and score_{deletion} are normalized as follows:

$$s c o r {e}_{mutation} = \frac{( s c o r {e}_{mutation} - min ( s {core}_{mutation} ) )}{( max ( s {core}_{mutation} ) - min ( s {core}_{mutation} ) )} \\ s c o r {e}_{deletion} = \frac{( s {core}_{deletion} - min ( s {core}_{deletion} ) )}{( max ( {score}_{deletion} ) - min ( {score}_{deletion} ) )}$$

computing the weights of scoreₘᵤₜₐₜᵢₒₙ and score_{deletion} as follows:

$$a = number of amino acids with deletion fold change > 1$$

$$b = number of amino acids with mutation fold change > 1$$

$${w}_{mutation} = \frac{a}{a + b}$$

$${w}_{deletion} = \frac{b}{a + b}$$

computing the essential score as follows:

essential score=w_{GHIJIKLM}*score_{GHIJIKLM}+W_{STUTIKLM}*score_{STUTIKLM}.

Finally, the amino acids are ranked based on their functional importance according to the essential scores.

### EXAMPLES

### Materials and Methods

### Cells and Reagents

Stably Cas9-expressing HeLa cells and HEK293T cells were cultured in Dulbecco's modified Eagle's medium (DMEM, Corning) containing 10% fetal bovine serum (FBS, CellMax) under 5% CO₂ at 37° C.

Plasmid Construction

The sgRNA vector (pLenti-sgRNA-GFP) was cloned by replacing the U6 promoter in pLL3.7 (Addgene) with the human U6 promoter, ccdB cassette and sgRNA scaffold. The Cas9 expression vector (pLenti-OC-IRES-BSD) has been previously reportedl. pcDNA-HBEGF was cloned by replacing the KRAB-dCas9 element of pHR-SFFVKRAB-dCas9-P2A-mCherry (Addgene) with the human HBEGF coding sequence and 3 ×FLAG. Vectors expressing cDNA of HBEGF with single amino acid deletions were constructed via PCR site-directed mutagenesis (PfuUltraII Fusion HS DNA Polymerase, STRATAGENE). The primers used to generate different deletion mutants for HBEGF are listed as follows.

(SEQ ID NO: 1)
HBEGF-29-F 5′-GACCGGAAAGTCCGTTTGCAAGAGGCAG-3′
(SEQ ID NO: 2)
HBEGF-29-R 5′-CTAGCCCTCTCCGCCGCTCCAGGCTC-3′
(SEQ ID NO: 1)
HBEGF-63-F 5′-GACCGGAAAGTCCGTTTGCAAGAGGCAG-3′
(SEQ ID NO: 3)
HBEGF-63-R 5′-CTGCCTCTTGCAAACGGACTTTCCGGTC-3′
(SEQ ID NO: 4)
HBEGF-70-F 5′-GCAAGAGGCAGATCTGCTTTTGAGAGTC-3′
(SEQ ID NO: 5)
HBEGF-70-R 5′-GACTCTCAAAAGCAGATCTGCCTCTTGC-3′
(SEQ ID NO: 6)
HBEGF-115-F 5′-CGGAAATACAAGGACTGCATCCATGGAG-3′
(SEQ ID NO: 7)
HBEGF-115-R 5′-CTCCATGGATGCAGTCCTTGTATTTCCG-3′
(SEQ ID NO: 8)
HBEGF-119-F 5′-GGACTTCTGCATCCATGAATGCAAATATGTG-3′
(SEQ ID NO: 9)
HBEGF-119-R 5′-CACATATTTGCATTCATGGATGCAGAAGTCC-3′
(SEQ ID NO: 10)
HBEGF-125-F 5′-GAATGCAAATATGTGGAGCTCCGGGCTCC-3′
(SEQ ID NO: 11)
HBEGF-125-R 5′-GGAGCCCGGAGCTCCACATATTTGCATTC-3′
(SEQ ID NO: 12)
HBEGF-127-F 5′-ATGTGAAGGAGCGGGCTCCCTCCTGC-3′
(SEQ ID NO: 13)
HBEGF-127-R 5′-GCAGGAGGGAGCCCGCTCCTTCACAT-3′
(SEQ ID NO: 14)
HEBGF-133-F 5′-GCTCCCTCCTGCTGCCACCCGGGTTAC-3′
(SEQ ID NO: 15)
HBEGF-133-R 5′-GTAACCCGGGTGGCAGCAGGAGGGAGC-3′
(SEQ ID NO: 16)
HEBGF-134-F 5′-CCCTCCTGCATCCACCCGGGTTACC-3′
(SEQ ID NO: 17)
HBEGF-134-R 5′-GGTAACCCGGGTGGATGCAGGAGGG-3′
(SEQ ID NO: 18)
HEBGF-138-F 5′-CTGCCACCCGGGTCATGGAGAGAGGTGTC-3′
(SEQ ID NO: 19)
HBEGF-138-R 5′-GACACCTCTCTCCATGACCCGGGTGGCAG-3′
(SEQ ID NO: 20)
HEBGF-141-F 5′-CCGGGTTACCATGGAAGGTGTCATGGGC-3′
(SEQ ID NO: 21)
HBEGF-141-R 5′-GCCCATGACACCTTCCATGGTAACCCGG-3′
(SEQ ID NO: 22)
HEBGF-152-F 5′-GCCTCCCAGTGGAACGCTTATATACCTATG-3′
(SEQ ID NO: 23)
HBEGF-152-R 5′-CATAGGTATATAAGCGTTCCACTGGGAGGC-3′
(SEQ ID NO: 24)
HEBGF-153-F 5′-CCTCCCAGTGGAAAATTTATATACCTATGACC-3′
(SEQ ID NO: 25)
HBEGF-153-R 5′-GGTCATAGGTATATAAATTTTCCACTGGGAGG-3

sgRNA Library Design

The hg19 CDS sequences of target genes were downloaded from the UCSC genome browser (https://genome.ucsc.edu/), and all potential sgRNAs with the NAG or NGG PAM sequence were designed using a homemade script to build the library.

Construction of the CRISPR/Cas9 sgRNA Library

Two libraries were constructed to include 1,236 and 3,712 sgRNAs targeting three drug-associated proteins and three toxin receptors, respectively. Array-based oligos encoding sgRNAs were synthesized and amplified via PCR with corresponding primers that included the BsmBI recognition site at the 5′ end. Those primers used for PCR amplification of the array-based oligos encoding sgRNAs (primer for amplifying sgRNA oligos targeting drug-associated proteins) are listed as follows.

Drug library F
(SEQ ID NO: 26)
5′-TTGTGGAAAGGACGAAACCG-3′
Drug library R
(SEQ ID NO: 27)
5′-TGCTGTCTCTAGCTCTACGT-3′
Toxin library F
(SEQ ID NO: 28)
5′-TCTTCATATCGTATCGTGCG-3′
Toxin library R
(SEQ ID NO: 29)
5′-TAGTCGCTAGGCTATAACGT-3′

The amplified DNA products were ligated into the vector using the Golden Gate method. The ligation mixture was then transformed into Transl-T1 competent cells (Transgen) to generate the plasmid library. The sgRNA plasmid library was subsequently transfected into HEK293T cells, together with two viral packaging plasmids, pVSVG and pR8.74 (Addgene), using the X-tremeGENE HP DNA transfection reagent (Roche). HeLa cells were then infected with a low MOI (˜0.3) of lentivirus, and EGFP⁺ cells were collected 48 hour after infection via FACS.

### Library Screening

For BI2536 and bortezomib screening, each experimental replicate consisted of two 150 mm dishes with 3.5×10⁶ cells each. The cells were treated with drugs at an appropriate concentration at 24 hour after seeding. For the first round of screening, the library cells were cultured with BI2536 at 4 ng/ml for 1.5 days or bortezomib at 4 ng/ml for 3 days, followed by culturing in fresh DMEM. The resistant cells were re-seeded and cultured for 5-10 days for a subsequent round of drug screening. For the second round of screening, the library cells were incubated with BI2536 at 5 ng/ml for 4 days or with bortezomib at 8 ng/ml for 5 days. For the third round of screening, the library cells were incubated with BI2536 at 6 ng/ml for 3 days. For 6-TG screening, a total of 1.8×10⁷ library cells were plated onto 150 mm Petri dishes at 3 x10⁶ cells per plate. Three plates of cells were grouped together as one replicate. The cells were treated with 6-TG at 250 ng/ml for 6 days, and surviving cells were re-seeded for growth and subjected to the next round of screening. For the second and third rounds, the library cells were incubated with 6-TG at 250 ng/ml and 300 ng/ml, respectively, for 4 days. For TcdB screening, four 150 mm dishes were plated with 3.5×10⁶ cells each as one experimental replicate. For each round of screening, the cells were treated with an appropriate concentration: 70 ng/ml for the first round and 100 ng/ml for the second and third rounds. The details of the HBEGF and ANTXR1 screening were the same as described in our previous report⁽¹⁾.

The resistant cells from each screening were collected for genomic DNA and total RNA extraction, followed by reverse transcription. The sgRNA coding regions and cDNAs of the targeted genes obtained through PCR amplification were then subjected to next-generation sequencing (NGS) analysis.

Identification of Candidate sgRNA Sequences

Genomic DNA was extracted from an appropriate number of library cells using the DNeasy Blood and Tissue kit (Qiagen). The appropriate number of library cells was different for different drug/toxin treatments: 6.25×10⁵ for ANTXR1, 3×10⁶ for CSPG4, 2.5×10⁵ for HBEGF, 1.75×10⁵ for HPRT1, 6.3×10⁵ for PLK1 and 3×10⁵ for PSMB5. sgRNA regions were amplified via 26 cycles of PCR using primers' annealing to the flanking sequences of the sgRNAs. The PCR products from each replicate were pooled and purified with DNA Clean & Concentrator-5 (Zymo Research Corporation), indexed with different barcodes (NEB #7370, #7335, #7500) and analyzed via NGS.

cDNA Preparation and Sequencing

Total RNA was extracted from the library cells using the RNAprep Pure Cell/Bacteria Kit (TIANGEN), and cDNA was synthesized using the Quantscript RT Kit (TIANGEN). A two-step method was employed to construct libraries for NGS. The first step consisted of PCR amplification of the cDNA (26 cycles; PrimeSTAR HS DNA Polymerase, Takara). The primers used for the different genes (Primer for cDNA amplification) are listed in Table 1:

| Gene | Primer | Sequence | SEQ ID NO. |
|---|---|---|---|
| ANTXR1 | F1^{ANTXR1} | 5′-AACAGCATCGGAGCGGAAA-3′ | SEQ ID NO: |
| (Transcript 1) | | | 30 |
| | R1^{ANTXR1} | 5′-TGGGCTTTATCACCACTCCTC-3′ | SEQ ID NO: |
| | | | 31 |
| ANTXR1 | F2^{ANTXR1} | 5′-AATAAAGGACCCGCGAGGAAG-3′ | SEQ ID NO: |
| (Transcript 3) | | | 32 |
| | R2^{ANTXR1} | 5′-TTTTCAGGAGTGTGCTGTCCG-3′ | SEQ ID NO: |
| | | | 33 |
| CSPG4 | F1^{CSPG4} | 5′-TCCCAGCTCCCAGGACTC-3′ | SEQ ID NO: |
| | | | 34 |
| | R1^{CSPG4} | 5′-GGGTGTTCTGAGTGTGCAGT-3′ | SEQ ID NO: |
| | | | 35 |
| | F2^{CSPG4} | 5′-AGAGAGCCACTGTGTGGATGC-3′ | SEQ ID NO: |
| | | | 36 |
| | R2^{CSPG4} | 5′-GGAAGTGTGCTCGCCGTCAG-3′ | SEQ ID NO: |
| | | | 37 |
| | F3^{CSPG4} | 5′-GGGCTCGTGCTGTTCTCAC-3′ | SEQ ID NO: |
| | | | 38 |
| | R3^{CSPG4} | 5′-GCACCAGGCATGGAAGCAAT-3′ | SEQ ID NO: |
| | | | 39 |
| HBEGF | F1^{HBEGF} | 5′-CGAAAGTGACTGGTGCCTCG-3′ | SEQ ID NO: |
| | | | 40 |
| | R1^{HBEGF} | 5′-GGTCCCAATGGCAGATCCCT-3′ | SEQ ID NO: |
| | | | 41 |
| HPRT1 | F1^{HPRT1} | 5′-AGGCGAACCTCTCGGCTTT-3′ | SEQ ID NO: |
| | | | 42 |
| | R1^{HPRT1} | 5′-CAATCCGCCCAAAGGGAAC-3′ | SEQ ID NO: |
| | | | 43 |
| PLK1 | F1^{PLK}1 | 5′-CTCTGCTCGGATCGAGGTCT-3′ | SEQ ID NO: |
| | | | 44 |
| | R1^{PLK1} | 5′-GATGCAGGTGGGAGTGAGG-3′ | SEQ ID NO: |
| | | | 45 |
| PSMB5 | F1^{PSMB5} | 5′-TTCCCCGACCCCCTTCAGTG-3′ | SEQ ID NO: |
| (Transcript | | | 46 |
| 1 and 3) | R1^{PSMB5} | 5′-AGGATGGGTCACTGTGTCCGT-3′ | SEQ ID NO: |
| | | | 47 |
| PSMB5 | F2^{PSMB5} | 5′-TGGCCGACCTCACTTCC-3′ | SEQ ID NO: |
| (Transcript 2) | | | 48 |
| | R2^{PSMB5} | 5′-AAGTAAAACAAATAGTCACCTCTGC-3′ | SEQ ID NO: |
| | | | 49 |

The coding sequence of CSPG4 was approximately 6.9 kb in length, and three amplification reactions were employed to obtain overlapping fragments (˜50 bp) encompassing its full length. The PCR products from each cDNA fragment were pooled together and purified (DNA Clean & Concentrator-5, Zymo Research Corporation). Then, 1 μg of cDNA from each gene was sheared to ˜250 bp using the Covaris S2 system. The resulting sheared product was purified and concentrated using the DNA Clean & Concentrator-5 kit (Zymo Research Corporation) and indexed with different barcodes (NEB #7370, #7335, #7500) for NGS analysis.

### Computational Methods for Identifying Functional Domains

The sequencing reads were mapped to the reference sequences of target genes using Bowtie2 2.3.2 and sorted using SAMtools 1.3.1. Next, we filtered the reads to retain those that carried only missense mutations or in-frame deletions. For fragments containing missense mutations, we computed the mutation ratio of each amino acid as follows:

$$mutation ratio = \frac{number of sequenced mutations of the amino acid}{total number of sequenced reads of the amino acid}$$

For fragments containing in-frame deletions, we computed the deletion ratio of each amino acid as follows:

$$deletion ratio = \frac{number of sequence deletions of the amino acid}{total number of sequence reads of the amino acid}$$

We then categorized the mutation types based on the number of amino acid deletions that they generated, and we classified them as either “driver deletions”, if they contained only single amino acid deletions, or “passenger deletions”, if they contained multiple amino acid deletions. After determining the mutation/deletion ratios and decoding the deletion patterns, the fold changes between the experimental and control groups were computed.

Next, the essential score for each amino acid was computed as follows: for the mutation fold change, a null distribution was built based on all fold changes, and scoreₘᵤₜₐₜᵢₒₙ=−log 10(P-value) was computed for each amino acid. For the deletion fold change, we first applied a tunable parameter, α, to weight the driver mutation and passenger mutation as follows:

deletion fold change=driver fold change+α*passenger fold change.

Subsequently, a null distribution was built via permutation 100 times, and score_{deletion}=−log10(P-value) was computed for each amino acid. Next, scoreₘᵤₜₐₜᵢₒₙ and score_{deletion} were normalized as follows:

$${score}_{mutation} = \frac{( {score}_{mutation} - min ( {score}_{mutation} ) )}{( max ( {score}_{mutation} ) - min ( {score}_{mutation} ) )}$$

$$s c o r {e}_{deletion} = \frac{( scor {e}_{deletion} - min ( scor {e}_{deletion} ) )}{( max ( scor {e}_{deletion} ) - min ( scor {e}_{deletion} ) )}$$

We then computed the weights of scoreₘᵤₜₐₜᵢₒₙ and score_{deletion} as follows:

$$a = number of amino acids with deletion fold change > 1$$

$$b = number of amino acids with mutation fold change > 1$$

$${w}_{mutation} = \frac{a}{a + b}$$

$${w}_{d e l etion} = \frac{b}{a + b}$$

Finally, the essential score was computed as follows:

essential score=w_{GHIJIKLM}*score_{GHIJIKLM}+w_{STUTIKLM}*score_{STUTIKLM}

### Validation of the Screening Results

For the validation of critical mutations of PSMB5 and PLK1, sgRNAs were designed near the mutation site, and each 119 nt ssODN donor encoded one amino acid substitution for a validated residue. All sgRNAs (sgRNA sequences for the validation of critical mutations) and ssODN donor sequences (ssODN donors encoded one amino acid substitution for a validated residue) are listed in Table 2 as follows.

| | Amino | | SEQ ID | | SEQ ID |
|---|---|---|---|---|---|
| Gene | acid | sgRNA | NO. | ssODN | NO. |
| PSMB5 | R78 | 5′-GTAA | SEQ ID | 5′-TTTTTGTGGTCTTATGTGGCCTGTTTTGTG | SEQ |
| | | GCACC | NO: 50 | TTTTCCTCTGATCTTAACAGTTCCGCCATG | NO: 61 |
| | | CGCTGT | | GAGTCATAGTTGCAGCTGACAGCAACGC | |
| | | AGCCC-3′ | | TACAGCGGGTGCTTACATTGCCTCCCAGA | |
| | | | | CG-3′ | |
| PSMB5 | T80 | 5′-GTAA | SEQ ID | 5′-TTTTTGTGGTCTTATGTGGCCTGTTTTGTG | SEQ ID |
| | | GCACC | NO: 50 | TTTTCCTCTGATCTTAACAGTTCCGCCATG | NO: 62 |
| | | CGCTGT | | GAGTCATAGTTGCAGCTGACAGCAGGGC | |
| | | AGCCC-3′ | | TGCCGCGGGTGCTTACATTGCCTCCCAGA | |
| | | | | CG-3′ | |
| PSMB5 | V90 | 5′-CTAT | SEQ ID | 5′-TTTCCTCTGATCTTAACAGTTCCGCCATG | SEQ ID |
| | | CACCTT | NO: 51 | GAGTCATAGTTGCAGCTGACTCCAGGGCT | NO: 63 |
| | | CTTCAC | | ACAGCGGGTGCTTACATTGCCTCACAGA | |
| | | CGTC-3′ | | CGGCCAAGAAGGTGATAGAGATCAACCC | |
| | | | | ATACC-3′ | |
| PSMB5 | M104 | 5′-CCTG | SEQ ID | 5′-AGATGCGTTCCTTATTTCGAAGCTCATA | SEQ ID |
| | | CTAGG | NO: 52 | GATTCGACATTGCCGAGCCAACAGCCGTT | NO: 64 |
| | | CACCAT | | CCCAGAAGCTGCAATCCGCTGCGCCGCCA | |
| | | GGCTG-3′ | | GCGATGGTGCCTAGCAGGTATGGGTTGAT | |
| | | | | CTCT-3′ | |
| PSMB5 | A108 | 5′-AATC | SEQ ID | 5′-ACTCCAGGGCTACAGCGGGTGCTTAC | SEQ ID |
| | | CGCTG | NO: 53 | ATTGCCTCCCAGACGGTGAAGAAGGTGA | NO: 65 |
| | | CGCCC | | TAGAGATCAACCCATACCTGCTAGGCACA | |
| | | CCAGC | | ATGGCTGGGGGCACCGCGGATTGCAGCT | |
| | | CA-3′ | | TCTGGGAA-3′ | |
| PSMB5 | D110 | 5′-GCGC | SEQ ID | 5′-CAGTTTGGAGGCAGCTGCTACAGAGAT | SEQ ID |
| | | AGCGG | NO: 54 | GCGTTCCTTATTTCGAAGCTCATAGATTC | NO: 66 |
| | | ATTGC | | GACATTGCCGAGCCAACAGCCGTTCCCA | |
| | | AGCTTC-3′ | | GAAGCTGCAGGCCGCTGCGCCCCCAGCC | |
| | | | | ATGGTGC-3′ | |
| PSMB5 | C111 | 5′-GCGC | SEQ ID | 5′-CAGTTTGGAGGCAGCTGCTACAGAGAT | SEQ ID |
| | | AGCGG | NO: 54 | GCGTTCCTTATTTCGAAGCTCATAGATTC | NO: 67 |
| | | ATTGC | | GACATTGCCGAGCCAACAGCCGTTCCCA | |
| | | AGCTTC-3′ | | GAAGCTGGCATCCGCTGCGCCCCCAGCC | |
| | | | | ATGGTGC-3′ | |
| PSMB5 | C122 | 5′-TCTG | SEQ ID | 5′-ATACACCATGTTGGCAAGCAGTTTGG | SEQ ID |
| | | GGAAC | NO: 55 | AGGCAGCTGCTACAGAGATGCGTTCCTT | NO: 68 |
| | | GGCTGT | | ATTTCGAAGCTCATAGATTCGGAATTGG | |
| | | TGGCT-3′ | | CGAGCCAACAGCCGTTCCCAGAAGCTGC | |
| | | | | AATCCGCTG-3′ | |
| PSMB5 | G242 | 5′-TCCA | SEQ ID | 5′-GCAGGCCTATGATCTGGCCCGTCGAG | SEQ ID |
| | | GCCATC | NO: 56 | CCATCTACCAAGCCACCTACAGAGATGC | NO: 69 |
| | | CTCCCG | | CTACTCAGGAGGTGCAGTCAACCTCTAT | |
| | | CACG-3′ | | CACGTGCGGGAGGATGACTGGATCCGAG | |
| | | | | TCTCCAGTG-3′ | |
| PSMB5 | Negative | 5′-TCTT | SEQ ID | 5′-CGCAGCCTCGCCCACCAGCACGTCGTAG | SEQ ID |
| | | AGCTG | NO: 57 | GATTCCACGGCTTTTTCGAGGACAACGACT | NO: 70 |
| | | ACTAC | | TCGTGTTCGTGGTGTTGGAGCTCTGTAGCA | |
| | | GCGTA | | GGGTGAGTGTCGCTGCTGGGGAACTGGAAC | |
| | | A-3′ | | T-3′ | |
| PLK1 | C67 | 5′-GTCC | SEQ ID | 5′-AAGAGATCCCGGAGGTCCTAGTGGACCC | SEQ ID |
| | | GAGAT | NO: 58 | ACGCAGCCGGCGGCGCTATGTGCGGGGCC | NO: 71 |
| | | CTCGA | | GCTTTTTGGGCAAGGGCGGCTTTGCAAA | |
| | | AGCAC | | GGTGTTCGAGATCTCGGACGCGGACACC | |
| | | T-3′ | | AAGGAG-3′ | |
| PLK1 | R136 | 5′-CAGC | SEQ ID | 5′-CAGCCTCGCCCACCAGCACGTCGTAGGA | SEQ ID |
| | | GACAC | NO: 59 | TTCCACGGCTTTTTCGAGGACAACGACTTC | NO: 72 |
| | | TCACCC | | GTGTTCGTGGTGTTGGAGCTCTGTAGGCG | |
| | | TCCGG-3′ | | GGGCGTGAGTGTCGCTGCTGGGGAACTG | |
| | | | | GAAC-3′ | |
| PLK1 | F183 | 5′-CCTT | SEQ ID | 5′-CTCCCAGCCTCCTCCAAATTCCAGCCT | SEQ ID |
| | | TTCCTG | NO: 60 | OCTTGTAGTGATGTCAAGCACCCCTGCAGG | NO: 73 |
| | | AATGA | | CTCAGCAACTCACCTATTTTCACCTCGAGAT | |
| | | AGATC-3′ | | CTTCATTCAGCAGAAGGTTGCCCAGCTTG | |
| | | | | AGG-3′ | |
| PLK1 | Negative | 5′-TCTT | SEQ ID | 5′-ACTCCAGGGCTACAGCGGGTGCTTAC | SEQ ID |
| | | AGCTG | NO: 57 | ATTGCCTCCCAGACGGTGAAGAAGGTGA | NO: 74 |
| | | ACTAC | | TAGAGATCAACCCATACCTGCTAGGCACA | |
| | | GCGTA | | ATGGCTGGGGGCGCGGATTGCAGCTTCT | |
| | | A-3′ | | GGGAACGG-3′ | |

HeLa cells were transfected with 1 μg of sgRNA and 2 μg of the ssODN donor in six-well plates. Fourteen days after transfection, 1.5×10⁵ cells were seeded in six-well plates 24 hour before drug selection. Cells were treated with drugs at the proper dosages for 72 hour: bortezomib (8 ng/ml); BI2536 (10 ng/ml). The genomes of drug-resistant cells were extracted using the TIANamp Genomic DNA Kit (TIANGEN).

The mutated loci were amplified using TransTaq DNA Polymerase High Fidelity (Transgen) and purified using a Universal DNA Purification Kit (TIANGEN). The primers (primers for amplification of mutated loci in PSMB5 gene) are listed in Table 3.

| Name of | | SEQ | |
|---|---|---|---|
| Primers | Sequence | ID NO. | Description |
| PSMB5-F1 | 5′-GTGTTTTTGTGGTCTTATGTGGCC-3′ | SEQ ID | For PCR |
| | | NO: 75 | amplification of |
| PSMB5-R1 | 5′-CATGTGGTTGCAGCTTAACTCAC-3′ | SEQ ID | sgRNA targeted |
| | | NO: 76 | region of PSMB5 |
| PSMB5-F2 | 5′-GATGTGAAGCTCGGGTGACATT-3′ | SEQ ID | gene locus for |
| | | NO: 77 | Sanger sequencing |
| PSMB5-R2 | 5′-TCAGCATTGACACCAAGCCCTTT-3′ | SEQ ID | (R78, T80, M104, |
| | | NO: 78 | A108). |
| PSMB5-F3 | 5′-CTGCTAACCTCATCTCCCTTTCCAG-3 | SEQ ID | For PCR |
| | | NO: 79 | amplification of |
| PSMB5-R3 | 5′-CAAGCAGCTGCATCCACCCTCTT-3 | SEQ ID | sgRNA targeted |
| | | NO: 80 | region of PSMB5 |
| | | | gene locus for |
| | | | Sanger sequencing |
| | | | (G242). |

PCR fragments were cloned into the pEASY-T5 Zero Cloning Kit (Transgen) for sequencing.

### Cytotoxicity Assay

Cells were seeded in 96-well plates 24 hour before drug or toxin treatment (5,000 cells for diphtheria toxin (DT) and 3,000 cells for bortezomib), and different concentrations of bortezomib or DT were added. Cells were incubated at 37° C. for 48 hour (DT) or 72 hour (bortezomib) before the addition of 1 mg/ml of MTT (3-[4,5 -dimethylthiazol-2-yl]-2,5 -diphenyltetrazolium bromide). Spectrophotometer readings at 570 nm were collected using BioTek Cytation5 (BioTek Instruments).

### Results

To test CRESMAS approach in mapping functional elements of proteins, we selected three genes encoding bacterial toxin receptors (ANTXR1, CSPG4 and HBEGF) and three genes encoding cancer drug targets (HPRT1, PLK1 and PSMBS) (Table 4 as follows).

| | | | | Critical a.a. or |
|---|---|---|---|---|
| | | | Size of | domain for |
| Selection | | Target gene | protein | target function |
| of screen | Drug/Toxin | (essentiality) | (a.a.) | (known) |
| Bacterial | Anthrax toxin | ANTXR1 (No) | 564 | 56-67 a.a., |
| toxin | | | | 154-160 a.a. |
| | TcdB of | CSPG4 (No) | 2,322 | 401-560 a.a. |
| | Clostridum | | | |
| | difficile | | | |
| | Diphtheria | HBEGF (No) | 208 | F115, L127, |
| | toxin | | | E141 |
| Cancer | 6-TG | HPRT1 (No) | 218 | NA |
| drug | BI2536 | PLK1 (Yes) | 603 | G63, C67, R136 |
| | Bortezomib | PSMB5 (Yes) | 263 | R78, A79, T80, |
| | | | | M104, A108, |
| | | | | C111, C122, |
| | | | | G242 |

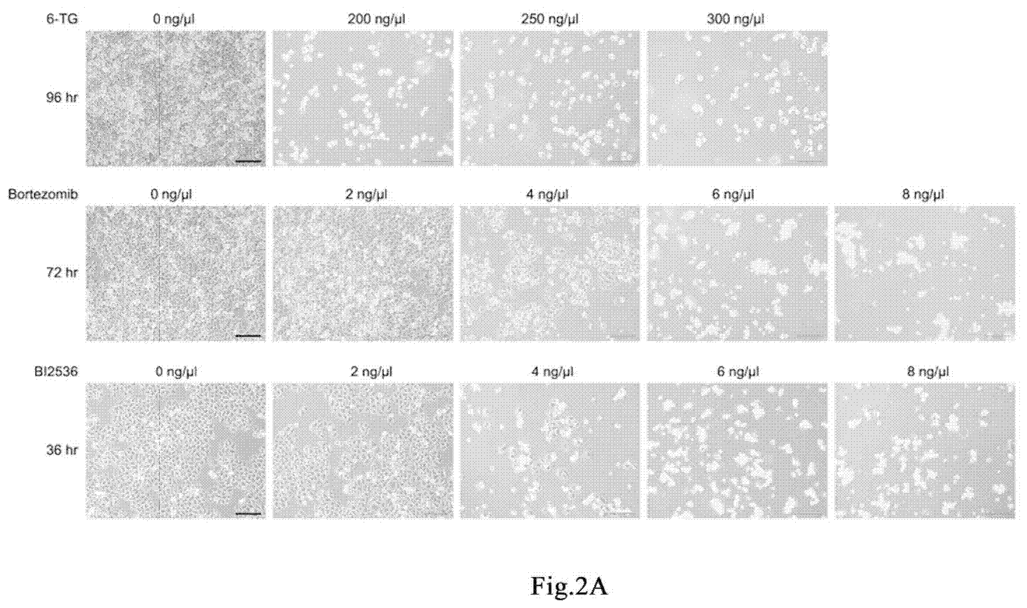

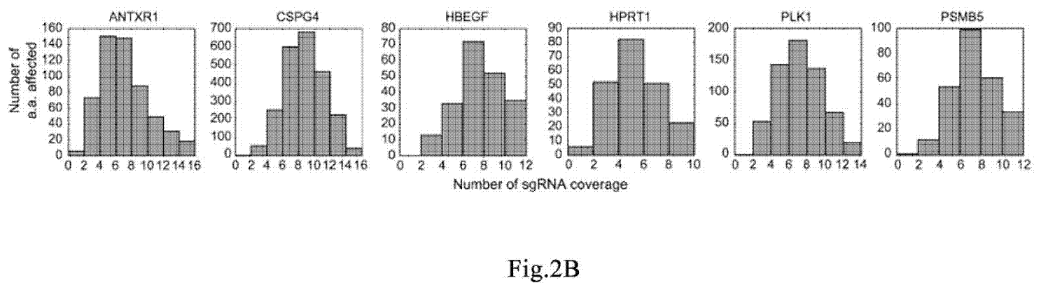

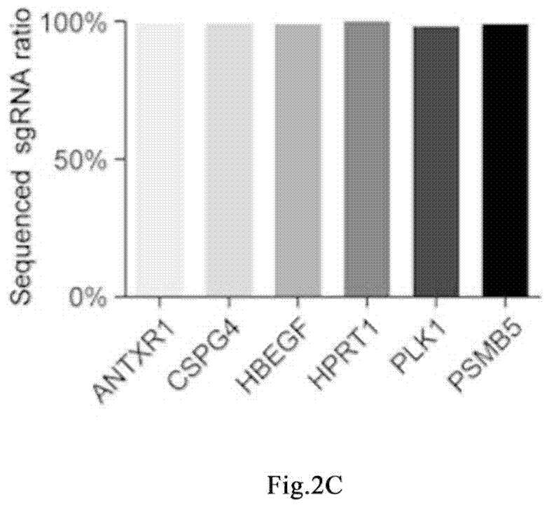

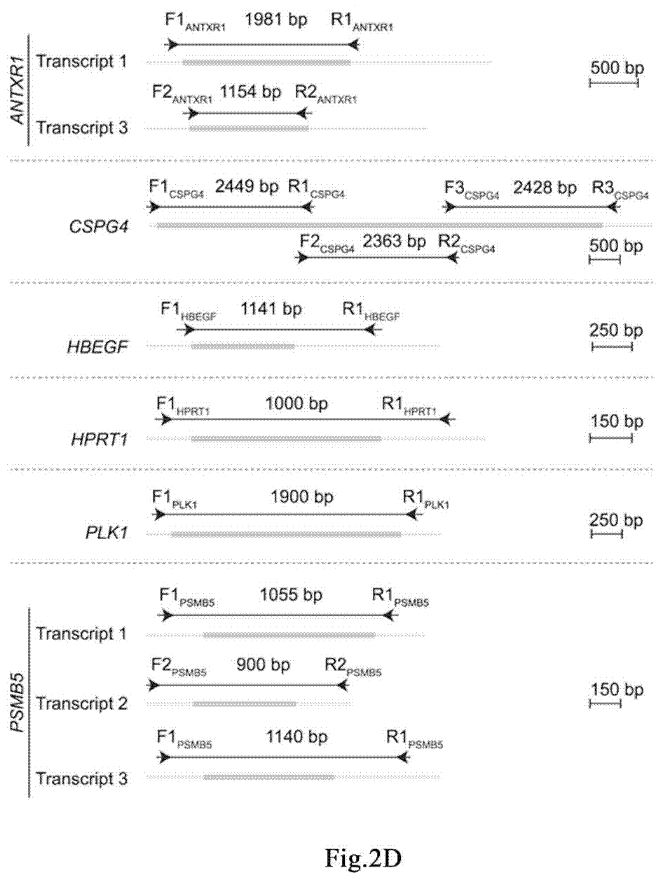

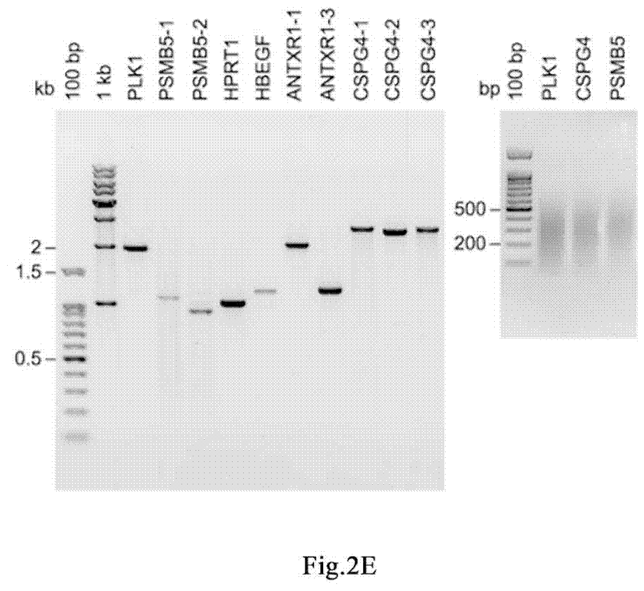

We chose HeLa cells to construct the CRISPR library for screening because we have determined the appropriate killing conditions in this line for toxins^{(8, 11)} and drugs, e.g., 6-TG (6-Thioguanine) targeting HPRT1⁽¹²⁾, BI2536 targeting PLK1⁽¹³⁾ and Bortezomib targeting PSMBS⁽¹⁴⁾ (FIG. 2A).

For targeted genes, sgRNAs were designed in silico and synthesized on a chip as pools to construct a saturation CRISPR library covering the full length of three receptor coding genes, and another library covering three drug targets (FIG. 2B).

We performed two replicates of functional screens for each of six treatments in addition to a control screen with no treatment. The sgRNA coverage of six genes was approximately 0.99 assuming that each sgRNA would affect 10-bp around the DSB site⁽¹⁵⁾ (FIG. 2C). After three rounds of toxin (PA/LFnDTA toxin, Diphtheria toxin or Clostridium difficile toxin B) or drug (6-TG BI2536 or Bortezomib) treatment, resistant cells were harvested and genome DNA was extracted for conventional sgRNA deciphering through NGS analysis^{(8, 16)}.

Meanwhile, these harvested resistant cells were subjected to total RNA isolation and reverse transcription to obtain cDNAs, which were subsequently used as templates for PCR amplification. Full length cDNAs of target genes were obtained through amplification using specific primers. For large-sized gene, such as CSPG4, three pairs of primers were used for amplification of three overlapping fragments in order to cover its full length. For genes with alternative splicing, specific primer pairs were designed to ensure all alternative transcripts were included (FIG. 2D and Table 1). Because of the size requirement for NGS, PCR fragments were further broken down to small sizes of average 250-bp (FIG. 2E). After all experimental procedures, we built a computational pipeline to analyze the sequencing data to identify amino acids essential for target gene function.

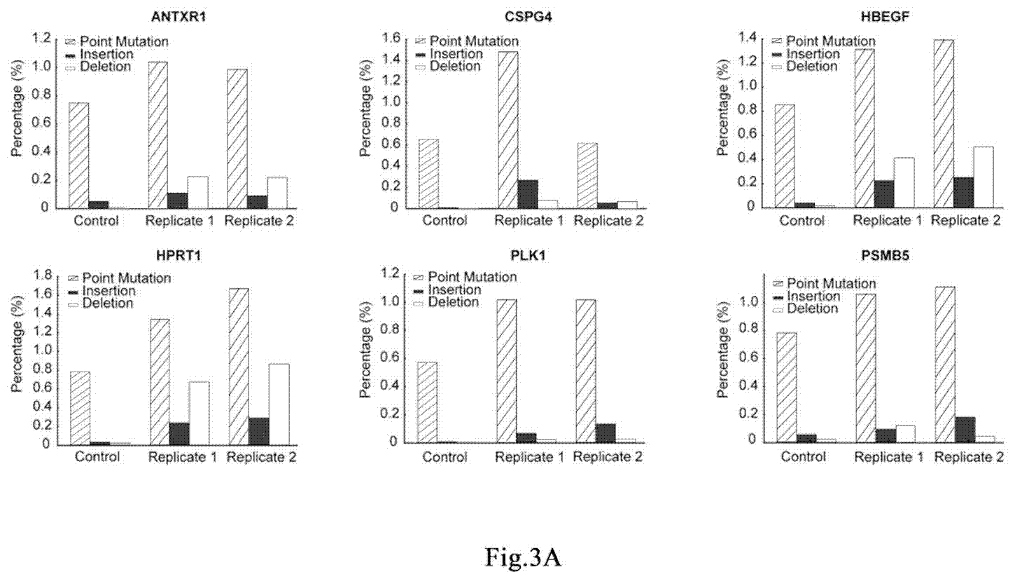

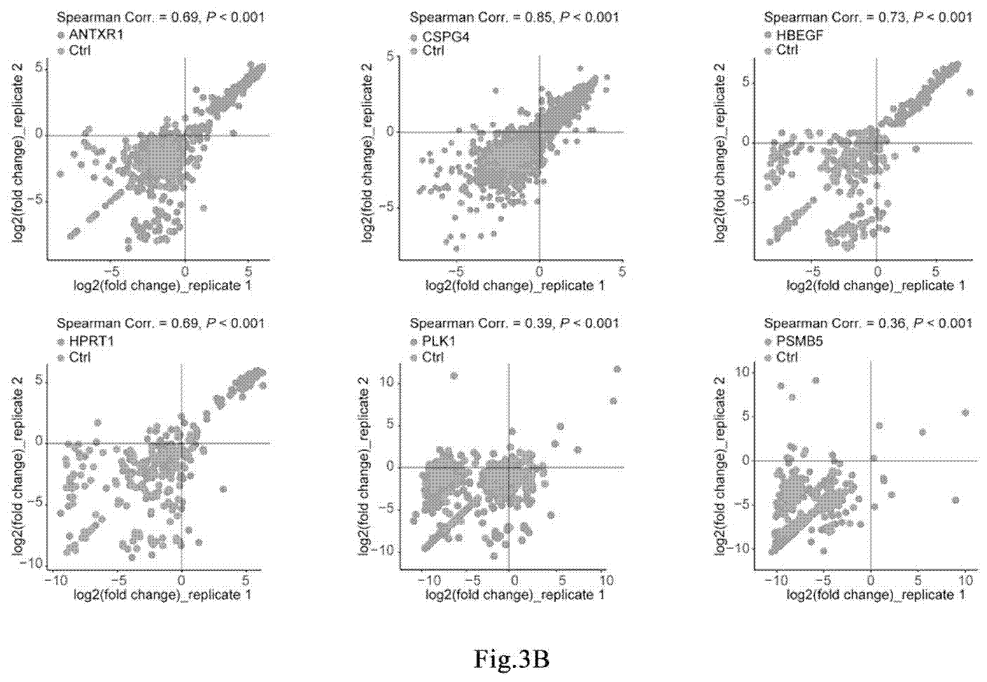

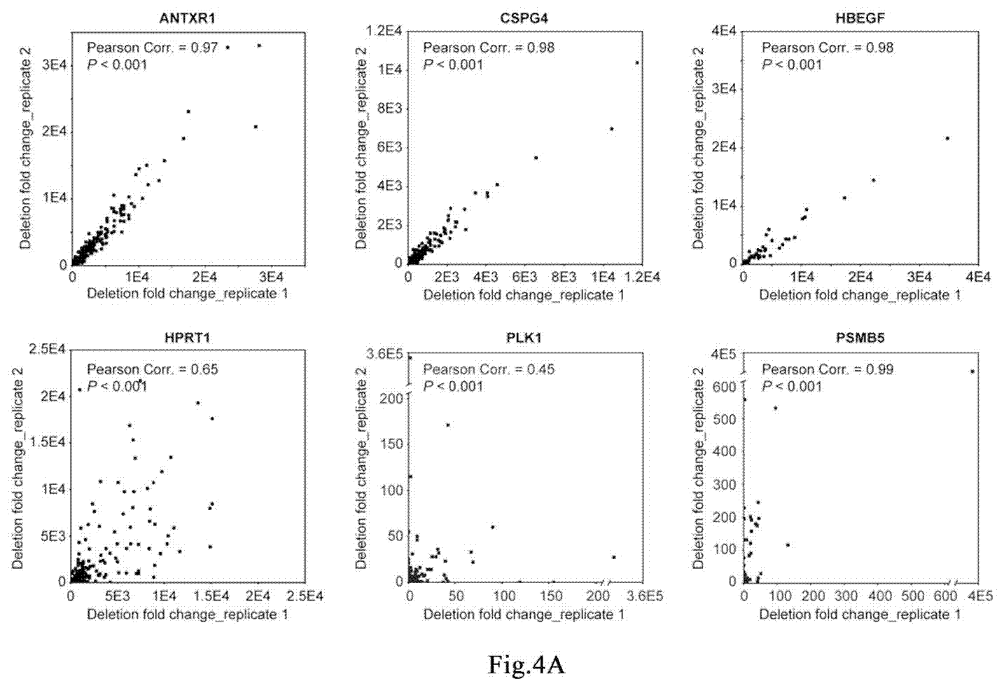

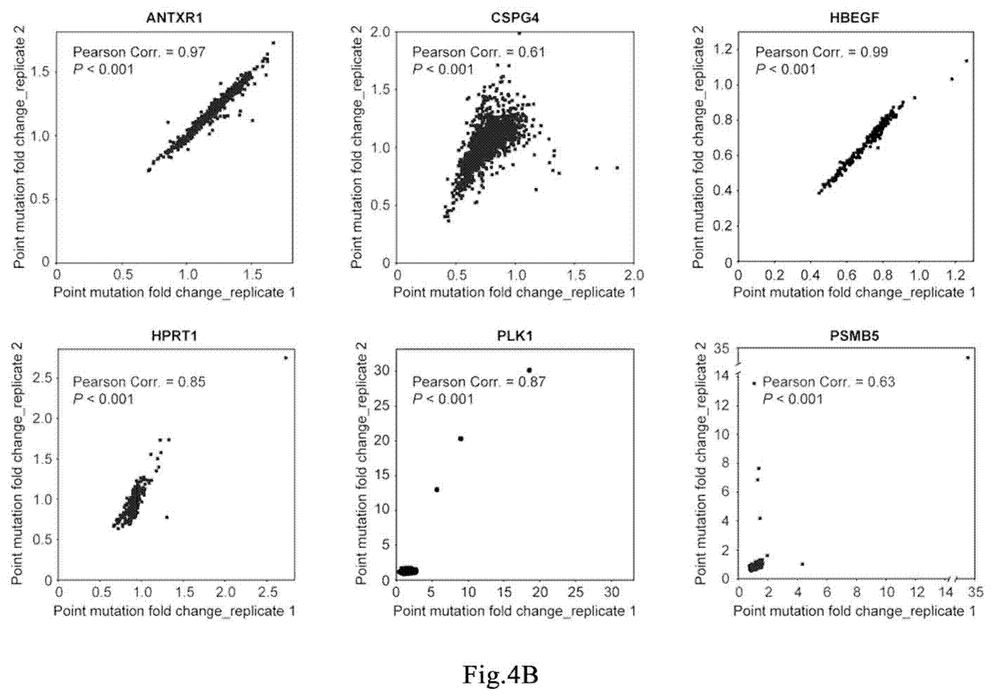

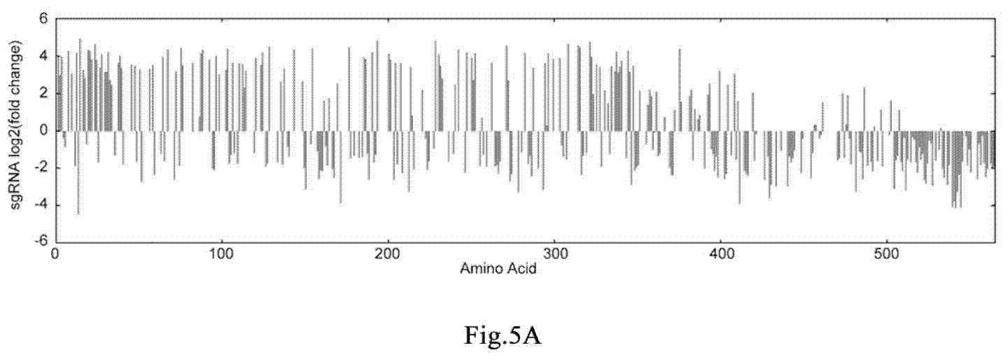

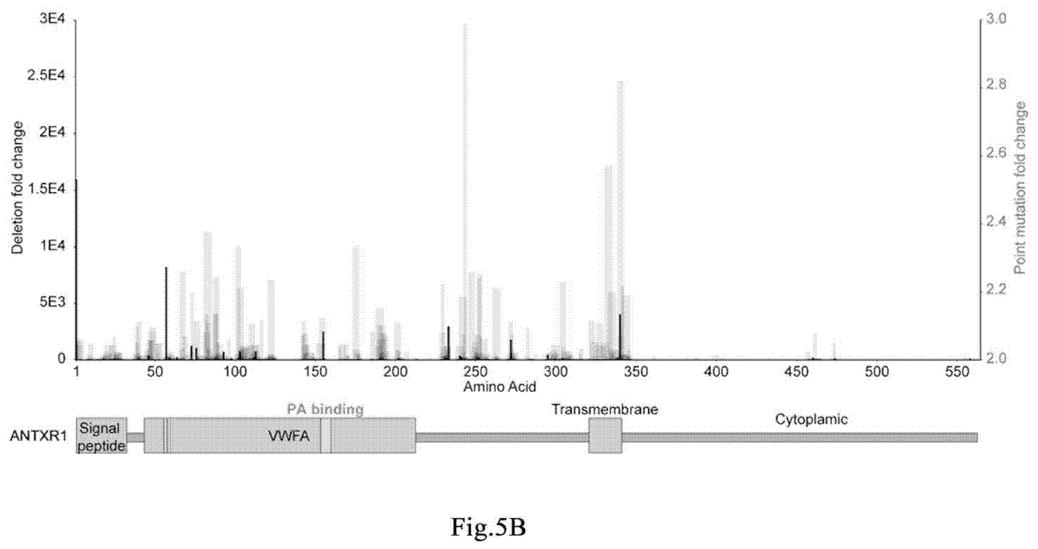

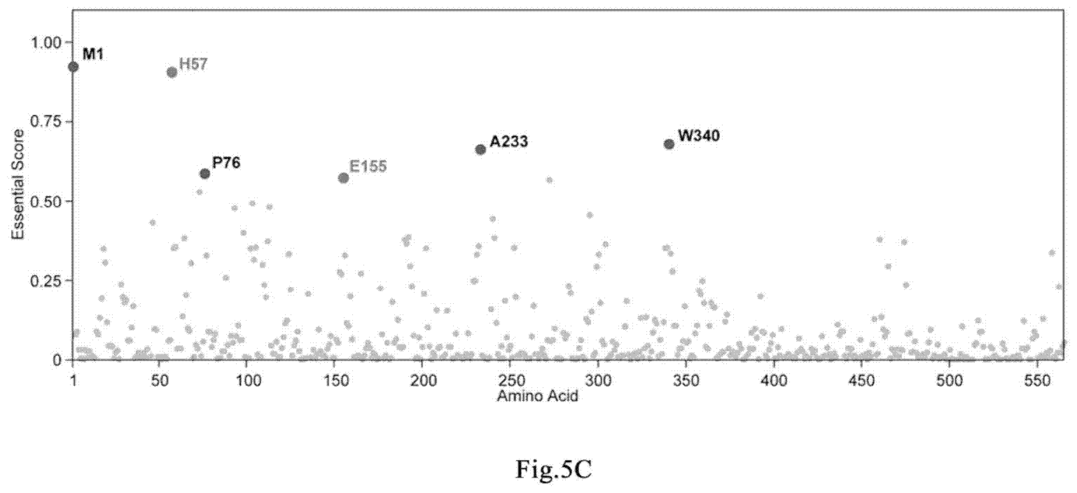

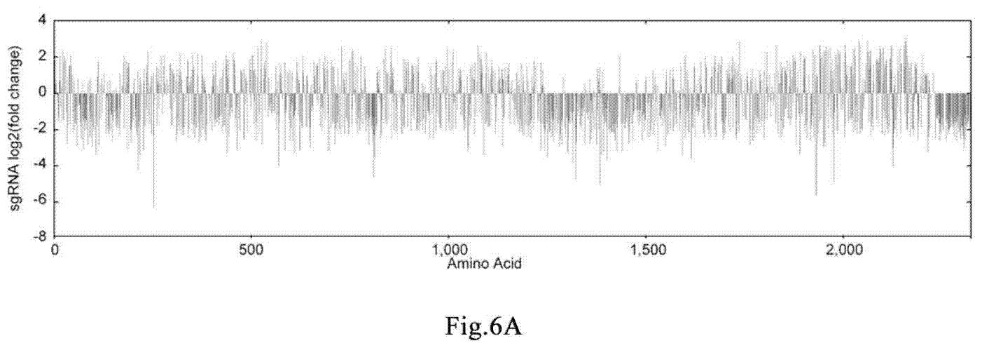

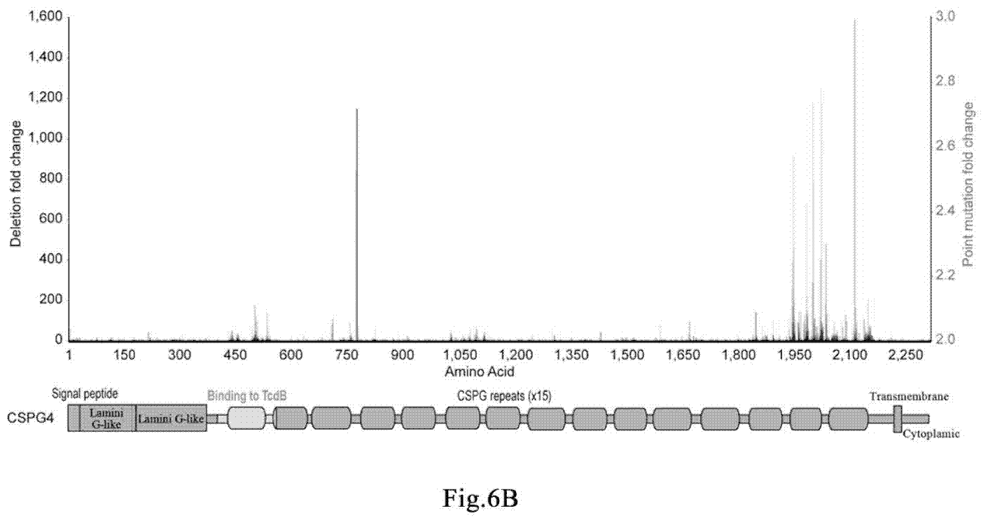

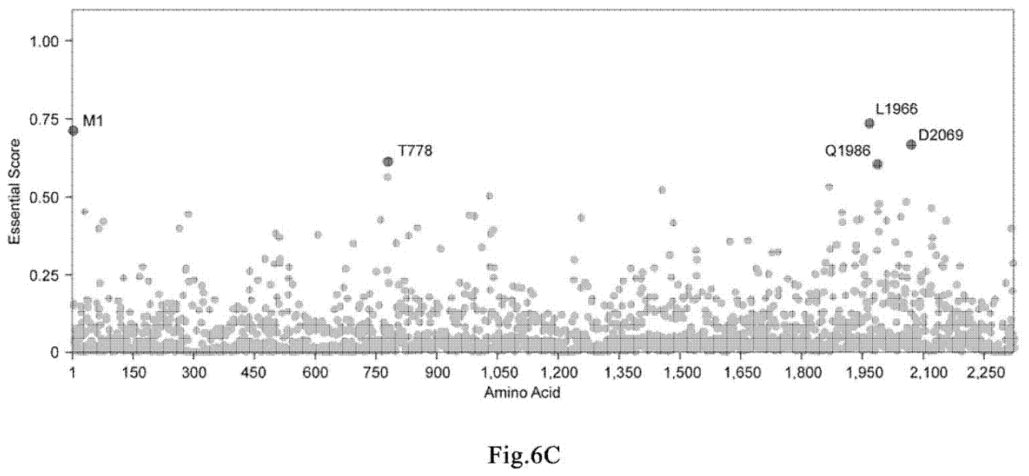

The percentages of mutations in control libraries were at low level for all six targets, and these numbers increased significantly after screening, especially the indels generated by CRISPR libraries. The relatively higher rates of point mutations in all controls were likely due to errors generated in PCR amplification and NGS. Nevertheless, reads of point mutation after all six screenings increased, suggesting certain point mutations did contribute to resistance phenotypes (FIG. 3A). We then evaluated the quality of screens through sgRNA fold changes between the two replicates and the correlation of deletion and point mutation ratios, and found that the correlation coefficient ranged from 0.36 to 0.85 for sgRNA fold change (FIG. 3B), 0.45 to 0.99 for deletion (FIG. 4A), and 0.61 to 0.99 for point mutation (FIG. 4), indicating the high consistency of our method. Because all three toxin receptors are nonessential for cell viability, their sgRNAs after screening were uniformly distributed across their coding sequences (FIG. 3A, FIG. 5A and FIG. 6A), indicating most of them were capable of generating frameshift indels, resulting in disruption of targeted gene expression. Interestingly, majority of their sgRNAs targeting coding regions corresponding to the C-terminal parts of three toxin receptors unanimously failed to get enriched (FIG. 3A, FIG. 5A and FIG. 6A), suggesting most of their intracellular C-terminal regions are functionally dispensable. Nevertheless, NGS of sgRNA-coding regions was incapable of revealing much sequence-to-function information.

Applying CRESMAS strategy with streamlined algorithms, we could obtain the function-related amino acid maps. We purposely assigned solid line to driver deletions because there is no ambiguity for the significance of this one-amino-acid-deletion type, while we assigned grey lines (10% scale) to those passenger deletions. We also merged the single missense mutation data with deletion data into one plot for easy visualization. Similar to single-amino-acid-deletion, loss of protein function due to missense point mutation demonstrated that the affected amino acid was essential for protein's function.

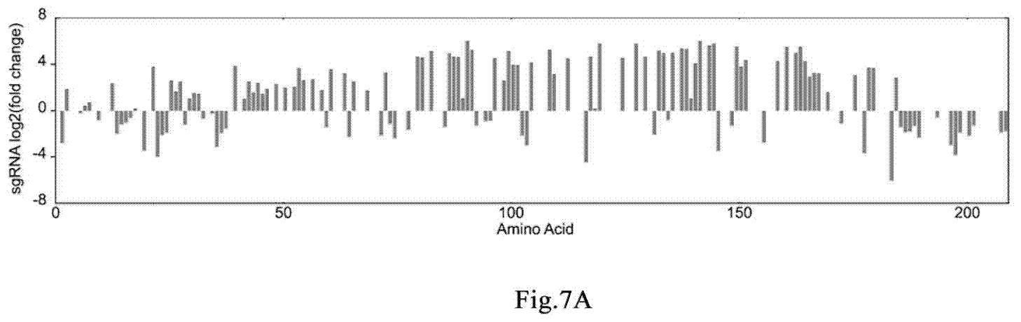

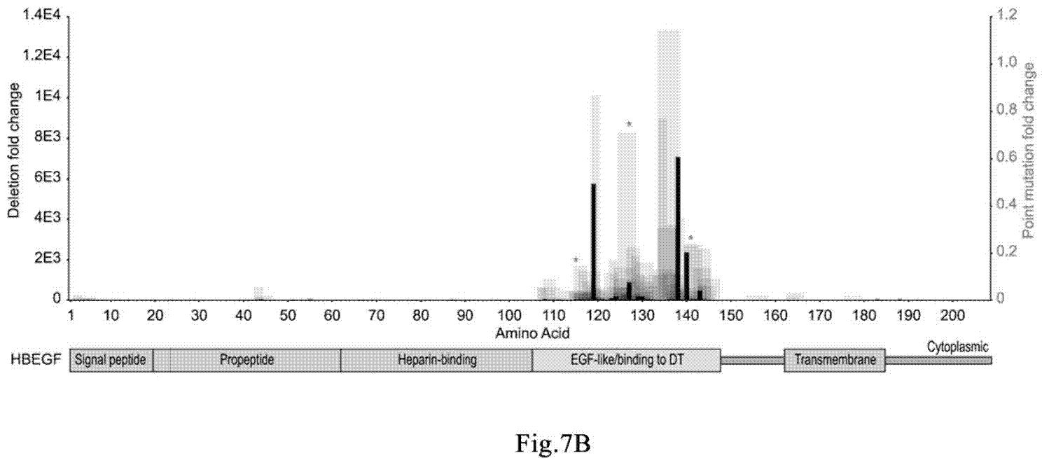

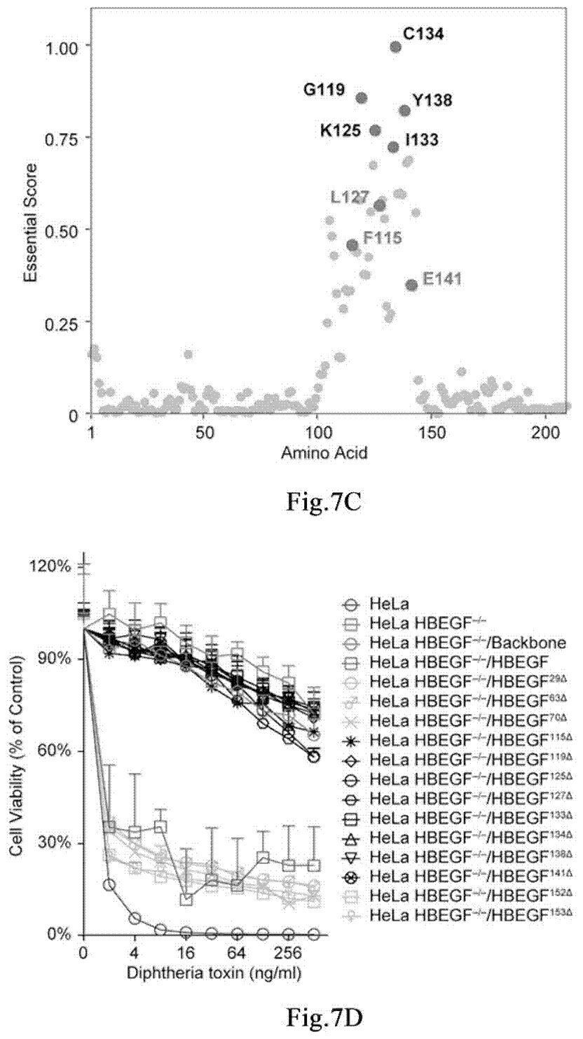

For the functional screening of HBEGF, which encodes a receptor for diphtheria toxin (DT), most of the resistant cells carried deletions in EGF-like domain (FIG. 7B), a reported DT-binding site⁽¹⁷⁾. Essential scores are computed and shown in Table 6 as follows.

| Amino | Essen | Amino | Essen | Amino | Essen |
|---|---|---|---|---|---|
| Acid | Score | Acid | Score | Acid | Score |
| 1 | 0.921289 | 151 | 0.062539 | 301 | 0.177932 |
| 2 | 0.077758 | 152 | 0.052577 | 302 | 0.059038 |
| 3 | 0.086672 | 153 | 0.276565 | 303 | 0.046487 |
| 4 | 0.030951 | 154 | 0.269416 | 304 | 0.363141 |
| 5 | 0.003633 | 155 | 0.572413 | 305 | 0.000961 |
| 6 | 0.0312 | 156 | 0.328178 | 306 | 0.005788 |
| 7 | 0.001443 | 157 | 0.115233 | 307 | 0.015109 |
| 8 | 0.028691 | 158 | 0.104132 | 308 | 0.05581 |
| 9 | 0.006644 | 159 | 0.199057 | 309 | 0.029554 |
| 10 | 0.027314 | 160 | 0.063618 | 310 | 0.046642 |
| 11 | 0.006079 | 161 | 0.006956 | 311 | 0.007768 |
| 12 | 0.010719 | 162 | 0.009137 | 312 | 0.005467 |
| 13 | 0.004849 | 163 | 0.011146 | 313 | 0.012518 |
| 14 | 0.088955 | 164 | 0.010824 | 314 | 0.011814 |
| 15 | 0.07926 | 165 | 0.271294 | 315 | 0.103653 |
| 16 | 0.130578 | 166 | 0.001678 | 316 | 0.18333 |
| 17 | 0.192124 | 167 | 0.013849 | 317 | 0.015036 |
| 18 | 0.349262 | 168 | 0.035756 | 318 | 0.000936 |
| 19 | 0.305694 | 169 | 0.051211 | 319 | 0.012339 |
| 20 | 0.116694 | 170 | 0.036975 | 320 | 0.017882 |
| 21 | 0.042397 | 171 | 0.004485 | 321 | 0.019732 |
| 22 | 0.044853 | 172 | 0.021169 | 322 | 0.002919 |
| 23 | 0.04109 | 173 | 0.014891 | 323 | 0.024174 |
| 24 | 0.004683 | 174 | 0.000763 | 324 | 0.130319 |
| 25 | 0.023049 | 175 | 0.002948 | 325 | 0.006415 |
| 26 | 0.028083 | 176 | 0.224824 | 326 | 0.034959 |
| 27 | 0.001495 | 177 | 0.07841 | 327 | 0.132617 |
| 28 | 0.238243 | 178 | 0.004323 | 328 | 0.043679 |
| 29 | 0.195796 | 179 | 0.013199 | 329 | 0.003153 |
| 30 | 0.178247 | 180 | 0.053144 | 330 | 0.024623 |
| 31 | 0.186536 | 181 | 0.001314 | 331 | 0.085095 |
| 32 | 0.059505 | 182 | 0.005609 | 332 | 0.124583 |
| 33 | 0.059277 | 183 | 0.181 | 333 | 0.112557 |
| 34 | 0.100536 | 184 | 0.052822 | 334 | 0.009904 |
| 35 | 0.168163 | 185 | 0.064335 | 335 | 0.061706 |
| 36 | 0.00512 | 186 | 0.124621 | 336 | 0.017791 |
| 37 | 0.008151 | 187 | 0.038382 | 337 | 0.117336 |
| 38 | 0.022264 | 188 | 0.036751 | 338 | 0.350896 |
| 39 | 0.008815 | 189 | 0.039762 | 339 | 0.353281 |
| 40 | 0.007937 | 190 | 0.377817 | 340 | 0.67822 |
| 41 | 0.022392 | 191 | 0.366091 | 341 | 0.335075 |
| 42 | 0.007437 | 192 | 0.385377 | 342 | 0.278946 |
| 43 | 0.032757 | 193 | 0.295004 | 343 | 0.106537 |
| 44 | 0.006877 | 194 | 0.230583 | 344 | 0.106189 |
| 45 | 0.010666 | 195 | 0.075909 | 345 | 0.014963 |
| 46 | 0.432089 | 196 | 0.002861 | 346 | 0.03399 |
| 47 | 0.095925 | 197 | 0.006228 | 347 | 0.036004 |
| 48 | 0.093355 | 198 | 0.068803 | 348 | 0.058405 |
| 49 | 0.009278 | 199 | 0.001086 | 349 | 0.167458 |
| 50 | 0.009091 | 200 | 0.038828 | 350 | 0.052496 |
| 51 | 0.000592 | 201 | 0.206937 | 351 | 0.05739 |
| 52 | 0.00868 | 202 | 0.350939 | 352 | 0.003421 |
| 53 | 0.009757 | 203 | 0.101272 | 353 | 0.012579 |
| 54 | 0.002353 | 204 | 0.041299 | 354 | 0.007356 |
| 55 | 0.059413 | 205 | 0.000986 | 355 | 0.081875 |
| 56 | 0.061114 | 206 | 0.020376 | 356 | 0.106963 |
| 57 | 0.904081 | 207 | 0.011871 | 357 | 0.21742 |
| 58 | 0.351311 | 208 | 0.155582 | 358 | 0.204816 |
| 59 | 0.355816 | 209 | 0.036448 | 359 | 0.247954 |
| 60 | 0.033665 | 210 | 0.040254 | 360 | 0.17757 |
| 61 | 0.035069 | 211 | 0.005573 | 361 | 0.040373 |
| 62 | 0.034171 | 212 | 0.006378 | 362 | 0.033457 |
| 63 | 0.135284 | 213 | 0.015866 | 363 | 0.106205 |
| 64 | 0.383144 | 214 | 0.153485 | 364 | 0.178173 |
| 65 | 0.202795 | 215 | 0.040539 | 365 | 0.165964 |
| 66 | 0.098151 | 216 | 0.040157 | 366 | 0.163801 |
| 67 | 0.090015 | 217 | 0.004259 | 367 | 0.004291 |
| 68 | 0.304371 | 218 | 0.004068 | 368 | 0.004816 |
| 69 | 0.004716 | 219 | 0.08122 | 369 | 0.016422 |
| 70 | 0.008457 | 220 | 0.014676 | 370 | 0.023599 |
| 71 | 0.045809 | 221 | 0.006153 | 371 | 0.02346 |
| 72 | 0.033796 | 222 | 0.007234 | 372 | 0.119106 |
| 73 | 0.529036 | 223 | 0.002215 | 373 | 0.141732 |
| 74 | 0.010153 | 224 | 0.00781 | 374 | 0.034062 |
| 75 | 0.055612 | 225 | 0.017701 | 375 | 0.013262 |
| 76 | 0.585654 | 226 | 0.082144 | 376 | 0.018157 |
| 77 | 0.32799 | 227 | 0.004551 | 377 | 0.023741 |
| 78 | 0.087957 | 228 | 0.016668 | 378 | 0.005824 |
| 79 | 0.086384 | 229 | 0.247671 | 379 | 0.021644 |
| 80 | 0.039652 | 230 | 0.248948 | 380 | 0.049295 |
| 81 | 0.061864 | 231 | 0.331271 | 381 | 0.034753 |
| 82 | 0.080595 | 232 | 0.357889 | 382 | 0.00052 |
| 83 | 0.003182 | 233 | 0.661655 | 383 | 0.001238 |
| 84 | 0.004518 | 234 | 0.012161 | 384 | 0.007194 |
| 85 | 0.005155 | 235 | 0.008635 | 385 | 0.017004 |
| 86 | 0.026239 | 236 | 0.00495 | 386 | 0.034225 |
| 87 | 0.025733 | 237 | 0.001011 | 387 | 0.084803 |
| 88 | 0.258091 | 238 | 0.00634 | 388 | 0.033432 |
| 89 | 0.045798 | 239 | 0.157889 | 389 | 0.096853 |
| 90 | 0.011092 | 240 | 0.442781 | 390 | 0.068293 |
| 91 | 0.074874 | 241 | 0.383787 | 391 | 0.001391 |
| 92 | 0.053676 | 242 | 0.115636 | 392 | 0.198336 |
| 93 | 0.477454 | 243 | 0.016835 | 393 | 0.087909 |
| 94 | 0.072754 | 244 | 0.002833 | 394 | 0.084606 |
| 95 | 0.107263 | 245 | 0.041855 | 395 | 0.014256 |
| 96 | 0.060908 | 246 | 0.003242 | 396 | 0.003602 |
| 97 | 0.062028 | 247 | 0.184554 | 397 | 0.031453 |
| 98 | 0.39954 | 248 | 0.069235 | 398 | 0.051013 |
| 99 | 0.00798 | 249 | 0.030231 | 399 | 0.076964 |
| 100 | 0.00568 | 250 | 0.043042 | 400 | 0.003818 |
| 101 | 0.005896 | 251 | 0.006265 | 401 | 0.002188 |
| 102 | 0.349741 | 252 | 0.352596 | 402 | 0.038386 |
| 103 | 0.493395 | 253 | 0.196369 | 403 | 0.0127 |
| 104 | 0.314871 | 254 | 0.013651 | 404 | 0.095579 |
| 105 | 0.353984 | 255 | 0.012398 | 405 | 0.005644 |
| 106 | 0.016101 | 256 | 0.019525 | 406 | 0.007074 |
| 107 | 0.00676 | 257 | 0.019219 | 407 | 0.009515 |
| 108 | 0.007114 | 258 | 0.014464 | 408 | 0.017435 |
| 109 | 0.299805 | 259 | 0.003542 | 409 | 0.009855 |
| 110 | 0.235559 | 260 | 0.003511 | 410 | 0.004453 |
| 111 | 0.195588 | 261 | 0.003572 | 411 | 0.008022 |
| 112 | 0.372971 | 262 | 0.072078 | 412 | 0.004036 |
| 113 | 0.481531 | 263 | 0.168776 | 413 | 0.022651 |
| 114 | 0.043335 | 264 | 0.016181 | 414 | 0.065987 |
| 115 | 0.019422 | 265 | 0.014325 | 415 | 0.033228 |
| 116 | 0.017175 | 266 | 0.003271 | 416 | 0.024776 |
| 117 | 0.055276 | 267 | 0.017973 | 417 | 0.00289 |
| 118 | 0.00465 | 268 | 0.033743 | 418 | 0.010931 |
| 119 | 0.00859 | 269 | 0.014119 | 419 | 0.005224 |
| 120 | 0.036676 | 270 | 0.001917 | 420 | 0.004917 |
| 121 | 0.071107 | 271 | 0.060375 | 421 | 0.033383 |
| 122 | 0.1135 | 272 | 0.565878 | 422 | 0.021286 |
| 123 | 0.123012 | 273 | 0.058195 | 423 | 0.028485 |
| 124 | 0.332336 | 274 | 0.06159 | 424 | 0.006799 |
| 125 | 0.220644 | 275 | 0.097638 | 425 | 0.000616 |
| 126 | 0.012103 | 276 | 0.003006 | 426 | 0.003036 |
| 127 | 0.044348 | 277 | 0.003301 | 427 | 0.073299 |
| 128 | 0.059597 | 278 | 0.001263 | 428 | 0.01051 |
| 129 | 0.0881 | 279 | 0.00181 | 429 | 0.01142 |
| 130 | 0.027129 | 280 | 0.084217 | 430 | 0.037141 |
| 131 | 0.000911 | 281 | 0.067185 | 431 | 0.016751 |
| 132 | 0.001783 | 282 | 0.076735 | 432 | 0.000496 |
| 133 | 0.002436 | 283 | 0.231922 | 433 | 0.007685 |
| 134 | 0.005362 | 284 | 0.209038 | 434 | 0.019628 |
| 135 | 0.206245 | 285 | 0.003849 | 435 | 0.007275 |
| 136 | 0.006567 | 286 | 0.001469 | 436 | 0.109582 |
| 137 | 0.005538 | 287 | 0.001111 | 437 | 0.076183 |
| 138 | 0.030466 | 288 | 0.003451 | 438 | 0.089329 |
| 139 | 0.004782 | 289 | 0.035848 | 439 | 0.08851 |
| 140 | 0.015944 | 290 | 0.060992 | 440 | 0.011255 |
| 141 | 0.094307 | 291 | 0.00966 | 441 | 0.003212 |
| 142 | 0.026068 | 292 | 0.000886 | 442 | 0.035817 |
| 143 | 0.014187 | 293 | 0.128379 | 443 | 0.015183 |
| 144 | 0.01339 | 294 | 0.117505 | 444 | 0.033089 |
| 145 | 0.006453 | 295 | 0.455059 | 445 | 0.003391 |
| 146 | 0.033381 | 296 | 0.150777 | 446 | 0.012045 |
| 147 | 0.047499 | 297 | 0.01131 | 447 | 0.005752 |
| 148 | 0.073985 | 298 | 0.020823 | 448 | 0.00442 |
| 149 | 0.006006 | 299 | 0.292619 | 449 | 0.062092 |
| 150 | 0.003911 | 300 | 0.331777 | 450 | 0.011365 |
| 451 | 0.010103 | 501 | 0.00216 | 551 | 0.006302 |
| 452 | 0.016919 | 502 | 0.000163 | 552 | 0.012947 |
| 453 | 0.000448 | 503 | 4.64E-05 | 553 | 0.128804 |
| 454 | 0.021766 | 504 | 0.000281 | 554 | 0.007478 |
| 455 | 0.009372 | 505 | 0.00014 | 555 | 0.022138 |
| 456 | 0.048329 | 506 | 0.016586 | 556 | 0.007396 |
| 457 | 0.127086 | 507 | 0.103799 | 557 | 0.027693 |
| 458 | 0.014819 | 508 | 0.000116 | 558 | 0.336684 |
| 459 | 0.018726 | 509 | 0.009611 | 559 | 0.006683 |
| 460 | 0.378648 | 510 | 6.96E-05 | 560 | 0.002242 |
| 461 | 0.133893 | 511 | 0.000328 | 561 | 0.021524 |
| 462 | 0.094774 | 512 | 0.000352 | 562 | 0.229858 |
| 463 | 0.072621 | 513 | 0.000376 | 563 | 0.020486 |
| 464 | 0.086148 | 514 | 0.045227 | 564 | 0.040766 |
| 465 | 0.294546 | 515 | 0.050857 | 565 | 0.054081 |
| 466 | 0.003331 | 516 | 0.121957 | | |
| 467 | 0.032521 | 517 | 0.086478 | | |
| 468 | 0.026765 | 518 | 0.087591 | | |
| 469 | 0.012823 | 519 | 0.040593 | | |
| 470 | 0.032246 | 520 | 0.000837 | | |
| 471 | 0.010771 | 521 | 0.001161 | | |
| 472 | 0.031976 | 522 | 0.001521 | | |
| 473 | 0.029329 | 523 | 0.0402 | | |
| 474 | 0.370677 | 524 | 0.033928 | | |
| 475 | 0.235764 | 525 | 0.010407 | | |
| 476 | 0.08083 | 526 | 0.011532 | | |
| 477 | 0.082251 | 527 | 0.000861 | | |
| 478 | 0.023321 | 528 | 0.00189 | | |
| 479 | 0.02493 | 529 | 0.000738 | | |
| 480 | 0.057346 | 530 | 0.050739 | | |
| 481 | 0.020158 | 531 | 0.032326 | | |
| 482 | 0.006491 | 532 | 0.004005 | | |
| 483 | 0.007727 | 533 | 0.0004 | | |
| 484 | 0.014051 | 534 | 0.001547 | | |
| 485 | 0.017612 | 535 | 0.002381 | | |
| 486 | 0.006916 | 536 | 0.00877 | | |
| 487 | 0.022915 | 537 | 0.000787 | | |
| 488 | 0.054246 | 538 | 0.010614 | | |
| 489 | 0.093727 | 539 | 0.013455 | | |
| 490 | 0.002804 | 540 | 0.000471 | | |
| 491 | 0.01352 | 541 | 0.034782 | | |
| 492 | 0.010254 | 542 | 0.120919 | | |
| 493 | 0.046589 | 543 | 0.032185 | | |
| 494 | 0.00252 | 544 | 0.03742 | | |
| 495 | 0.009184 | 545 | 0.000568 | | |
| 496 | 0.010003 | 546 | 0 | | |
| 497 | 0.015634 | 547 | 0.06634 | | |
| 498 | 0.000424 | 548 | 0.088198 | | |
| 499 | 0.000257 | 549 | 0.073901 | | |
| 500 | 0.030706 | 550 | 0.005052 | | |

By computing the essential scores (Table 6), we found that the amino acids with the highest scores were indeed enriched in the EGF-like domain, further confirmed the essentiality of this domain in mediating toxin binding. The three known amino acids essential for DT-HBEGF interaction, F115, L127 and E141⁽¹⁷⁾, were top ranked (21th, 15th and 28th) among all amino acids. Importantly, CRESMAS approach revealed a number of novel sites besides these three that appeared important for receptor function (FIG. 7C). To validate our results, we expressed wild-type or mutant HBEGF cDNA in HeLa HBEGF−/− cells⁽⁸⁾ via lentiviral infection. We verified five top ranking sites (G119, K125, 1133, C134, Y138), three known positive sites and five low ranking sites (L29, D63, D70, N152, R153). HeLa HBEGF−/− appeared total resistant to DT, and the wild-type HBEGF expression could recover cell sensitivity to the toxin. All mutant HBEGF expression containing single amino acid deletion of one of these five top ranking sites (G119, K125, 1133, C134, Y138) or known positive sites (F115, L127, E141) failed to rescue sensitivity of cells to DT, while mutant HBEGF with deletion of either one of the five low ranking sites (L29, D63, D70, N152, R153) made the rescue just like the wild-type (FIG. 7D). These results confirmed our screening results that certain amino acids in the EGF-like domain are essential for DT-triggered cytotoxicity. Of note, the fact that few amino acids out of the DT-binding domain were screened out for HBEGF indicated that CRESMAS has low false positive rate.

For anthrax toxin's receptor, ANTXR1, all resistant cells carried variety of deletions across the whole coding region except that encoding the cytoplasmic domain (FIG. 5B and 5C), indicating that the interaction between anthrax toxin and ANTXR1 was dominated by the receptor's extracellular region. In addition to the known PA-binding sites⁽¹⁸) and transmembrane domain, a number of novel amino acids were identified that showed variable levels of importance (FIG. 5B). Consistent with sgRNA sequencing results (FIG. 5A), most amino acids within the cytoplasmic region were dispensable (FIG. 5B), again suggesting a low false positive rate for CRESMAS. The top amino acids critical for ANTXR1 function in mediating anthrax toxicity were determined by computing essential scores, including two known sites H57 and E155⁽¹⁸⁾ (FIG. 5C).

For CSPG4, the receptor of Clostridium difficile toxin B (TcdB), the peaks of mutants were mainly located in the first and last two CSPG repeats (FIGS. 6B and 6C). The first CSPG repeat was a known TcdB binding site⁽¹¹⁾, and the last two repeats were novel findings. Importantly, unlike the above two cases with HBEGF and ANTXR1 that most of the informative data were from deletion mutations, there was a missense point mutation affecting T778 in CSPG4 that was highly enriched (FIG. 6B), suggesting this very amino acid is critical for the receptor to mediate TcdB toxicity.

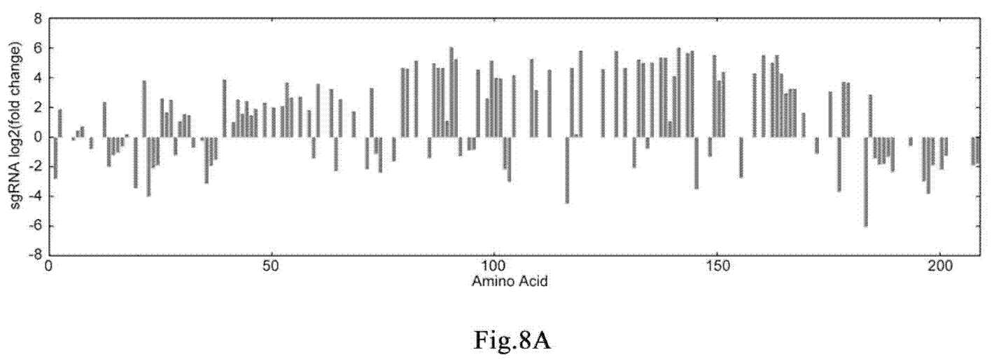

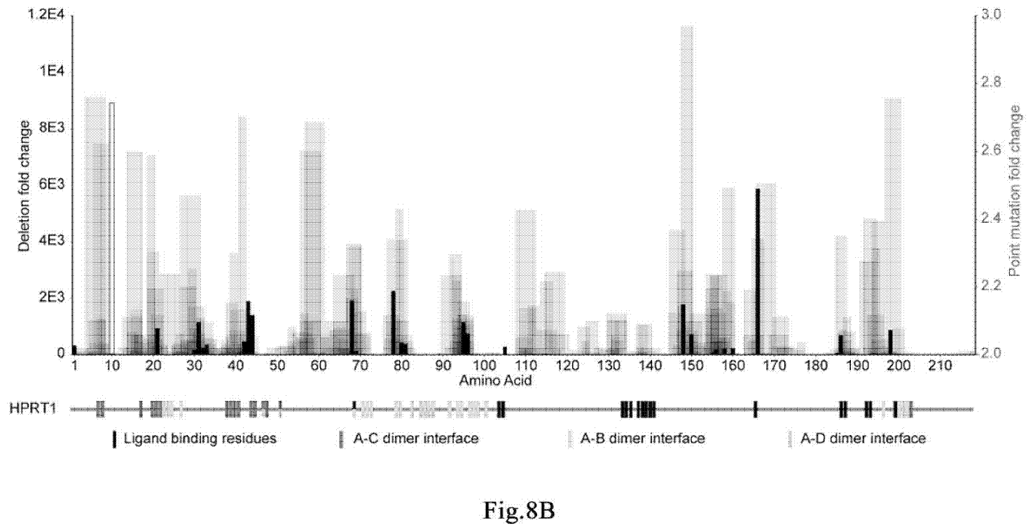

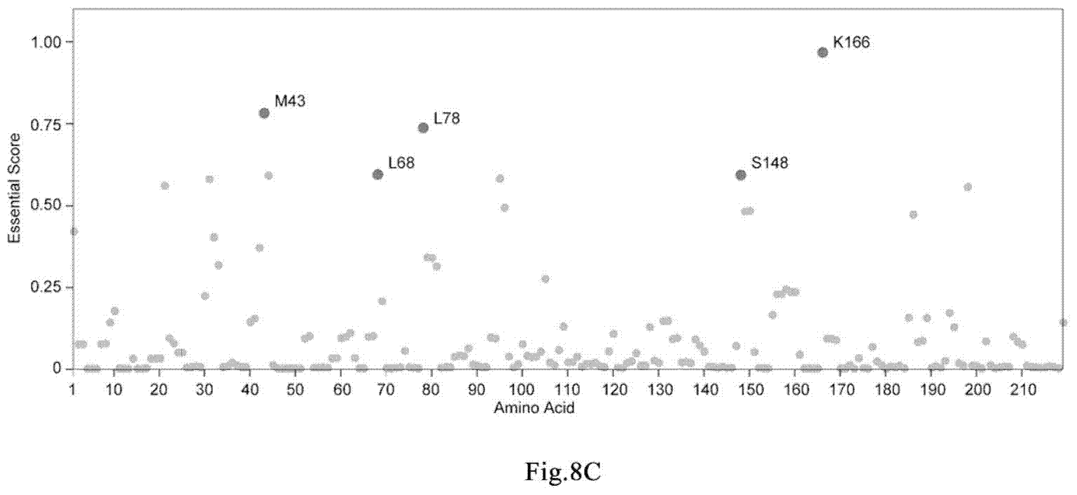

As for the three genes encoding cancer drug targets, HPRT1 is a nonessential gene, while PLK1 and PSMB5 are two essential genes⁽¹⁹⁾. For nonessential target HPRT1, 6-TG screening of the library showed that most of sgRNAs were enriched and evenly distributed (FIG. 8A), a result similar to those from the bacterial toxin screens (FIG. 3A, 5A, 6A). The significant role of each amino acid throughout the protein was completely buried. CRESMAS approach revealed that there existed numerous sites important for HPRT1 function in mediating cell sensitivity to 6-TG (FIG. 8B). This observation was consistent with the known structure of tetrameric HPRT1, and the sites with high essential score were also uniformly distributed (FIG. 8C)⁽¹²⁾.

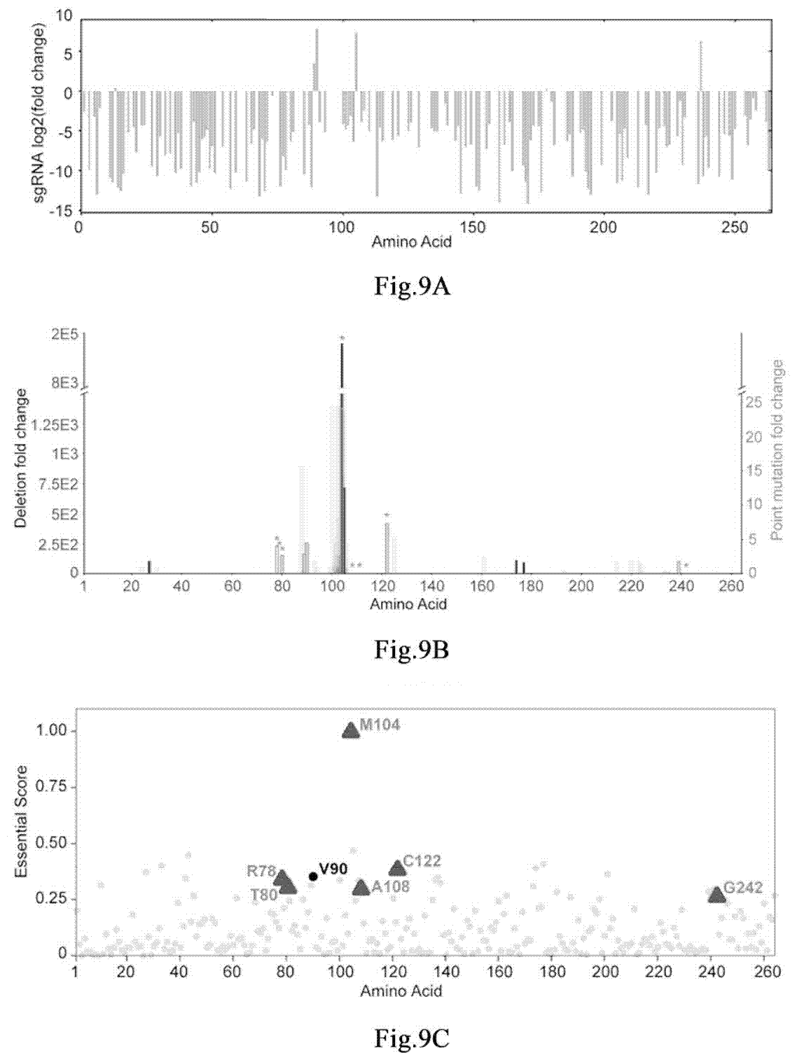

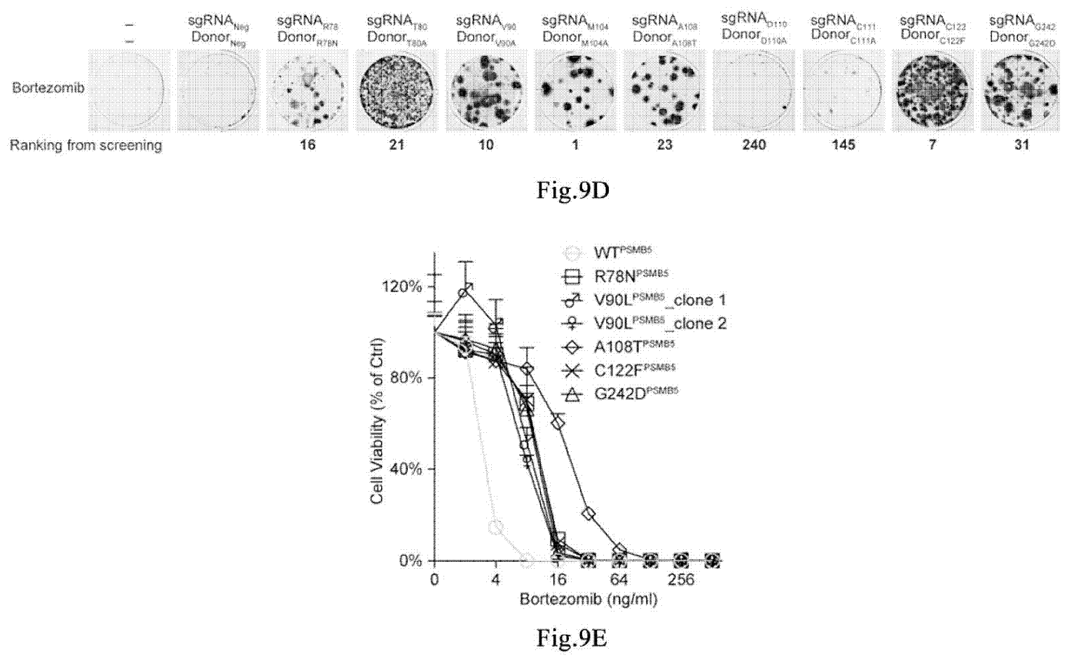

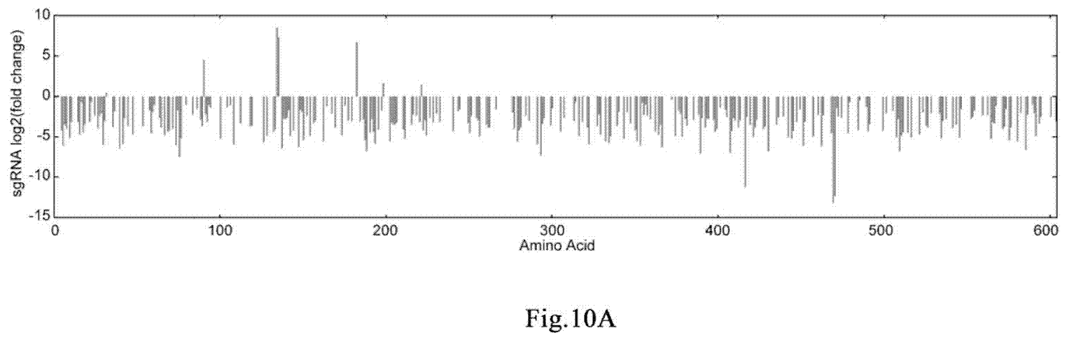

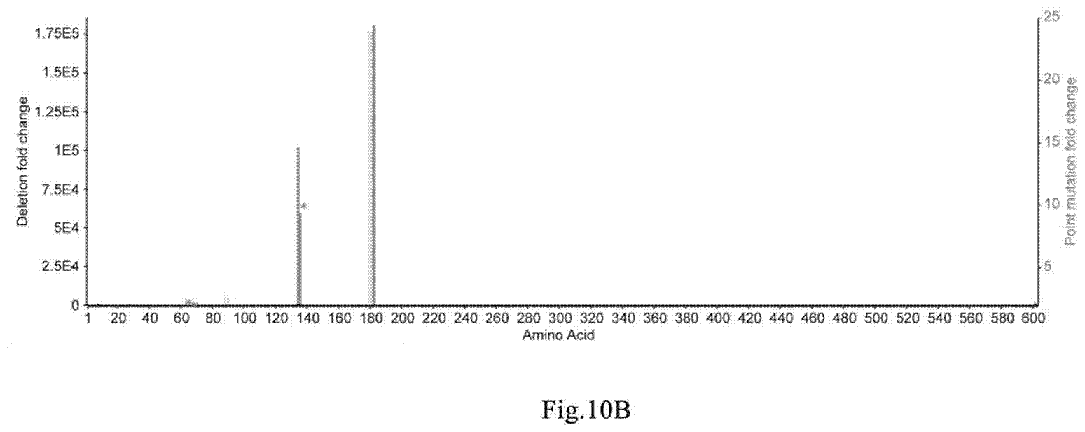

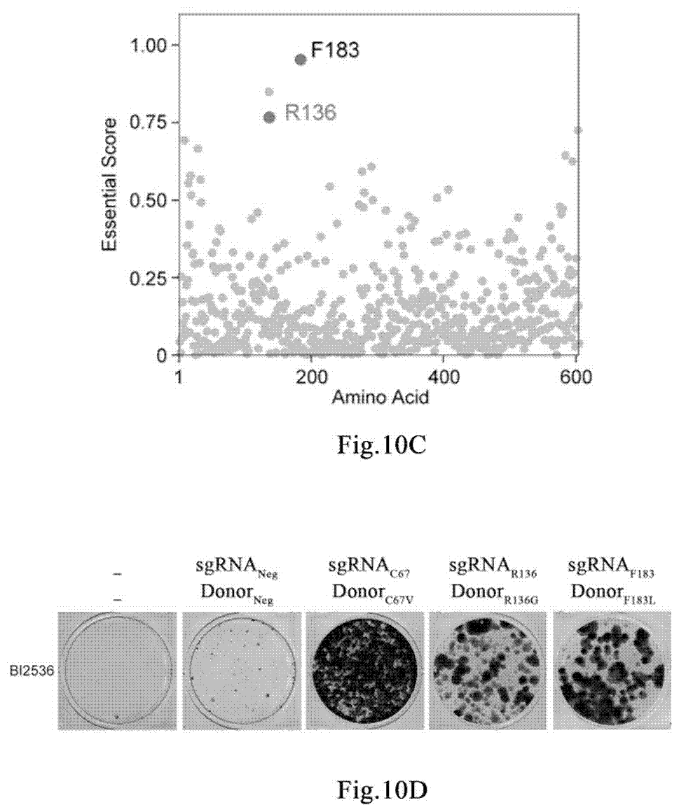

For essential targets, PLK1 and PSMB5, sgRNA sequencing did provide the approximate locations of certain critical amino acids where sgRNAs generated in-frame mutations (FIG. 9A and FIG. 10A). Because sgRNA enrichment provided indirect evidence and the resolution was low, we reasoned that CRESMAS strategy would reveal more precise and comprehensive map in more details. Indeed, more amino acids were identified with high accuracy in both PSMB5 and PLK1 that appeared critical for protein functions (FIG. 9B and FIG. 10B). Of note, the final screening results contained both missense mutations and variable number of deletions, and the top essential amino acids were obtained for both cases based on essential scores (FIG. 9C and FIG. 10C). Again, we identified both known critical sites in PSMB5 for its interaction with Bortezomib (R78, T80, M104, A108, C122 and G242) (20-22) and novel essential residues (FIG. 9B-C). Similarly, we identified the known residue R136 critical for BI2536-PLK1 interaction (22, 23) and a novel essential residue F183 (FIG. 10B-C).

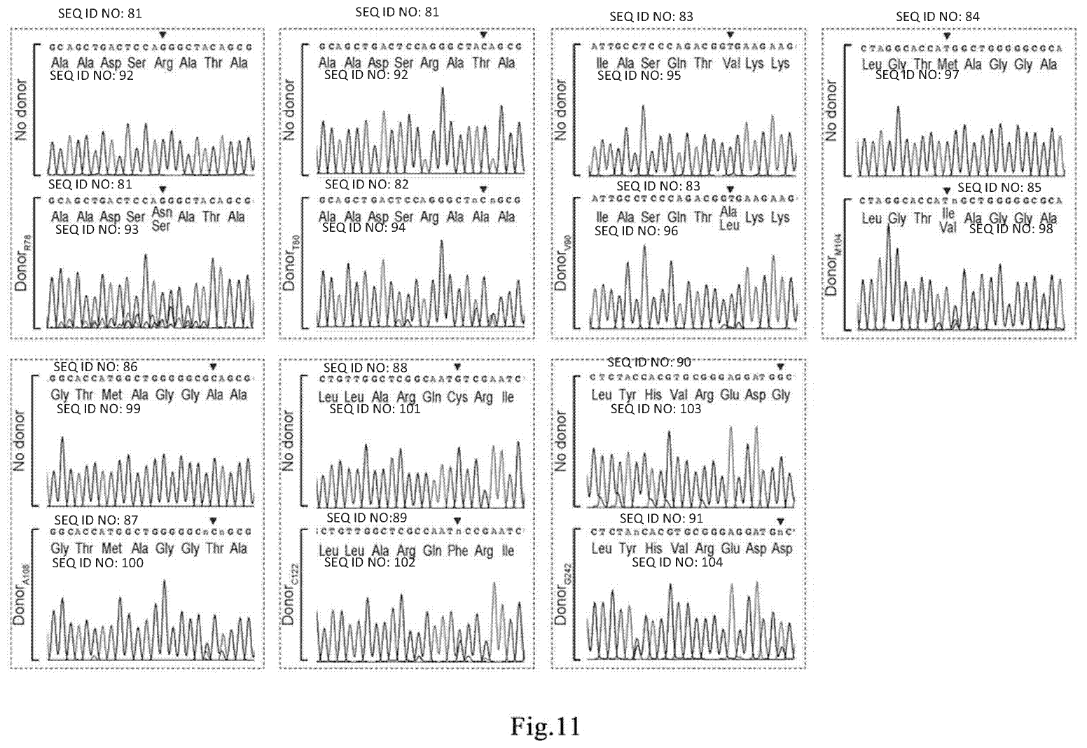

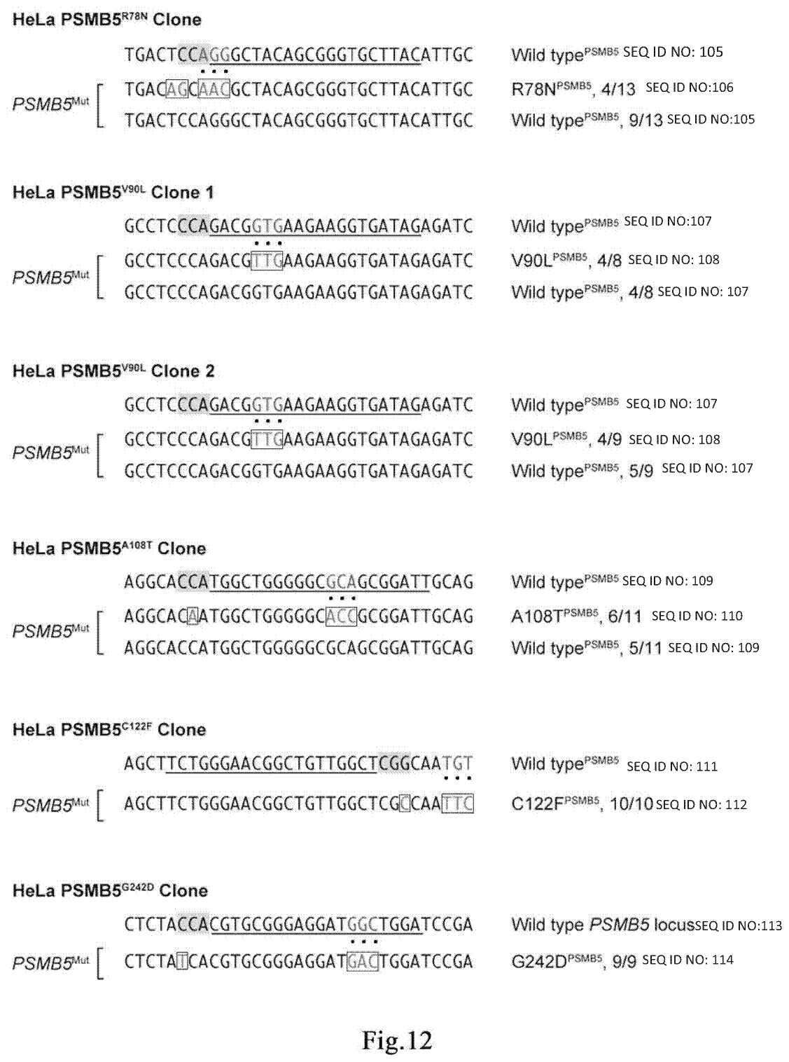

Because missense point mutations were the predominant formats conferring drug resistance for both PSMB5 and PLK1, we decided to employ ssODN-mediated method⁽²⁴⁾ to create specific point mutations instead of deletions for validation. We selected nine amino acid residues (R78, T80, V90, M104, A108, D110, C111, C122 and G242) in PSMB5, among which D110 and C111 were included as controls. To choose a proper amino acid for point mutation, the mutant types from screening results or previous reports were preferential choices. For the rest, we made all the substitution to alanine (Table 2). Cells transfected with donors containing one of the following mutations, R78N, T80A, V90A, M104A, A108T, C122F and G242D, produced variable number of Bortezomib resistant colonies (FIG. 9D). In comparison, D110A and C111A failed to produce Bortezomib resistant colonies, demonstrating that our method of validation was reliable (FIG. 9D). Interestingly, C111 site has previously been reported important for PSMB5 in SW1573 and CEM (21, 25), which is different from our screening and validation results (FIG. 9D). This discrepancy suggests either that the roles of amino acids are affected by biological contexts, or we failed to create the right amino-acid substitution to give rise to resistance phenotype. To verify the Bortezomib-resistant pooled cells, we sequenced the genomic region of targeted loci and confirmed that all these seven sites contained expected mutations (FIG. 11 and Table 3). To further verify our results, we isolated single clones from several mutant pools (FIG. 12) and performed cell viability assay. We demonstrated that the following point mutations conferred Bortezomib resistance, R78N, V9OL, A108T, C122F and G242D (FIG. 9E). Among them, T80 and A108 were reported involved in the direct binding of PSMB5 to Bortezomib⁽²⁰⁻²²⁾, and the mutations of R78, M104 and C122 were reported to confer Bortezomib resistance by disrupting drug-binding site structure^{(22, 26, 27)}. G242 was another known site related to Bortezomib sensitivity although the mechanism was not clear⁽²⁷⁾. V90 site was a novel finding. We picked two independent V90L clones, and both of them conferred drug resistance. It remains to be determined how V90 mediates drug sensitivity and whether V90 alteration changes the structure around Bortezomib binding pocket.

For PLK1, we validated two top ranking residues (R136 and F183) and one potential false negative site (C67). It has been reported R136 is a critical amino acid for BI2536 and F183 is structurally important when PLK1 binds to BI2536^{(22, 23)}. Point mutation on either one of these three sites conferred BI2536 resistance in the pooled assay (FIG. 10D).

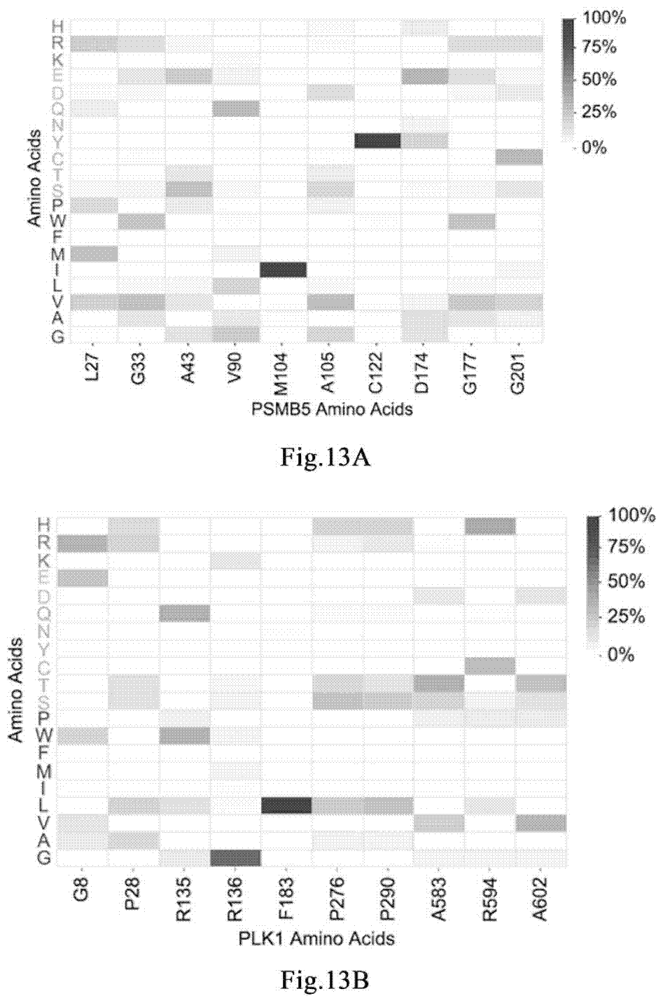

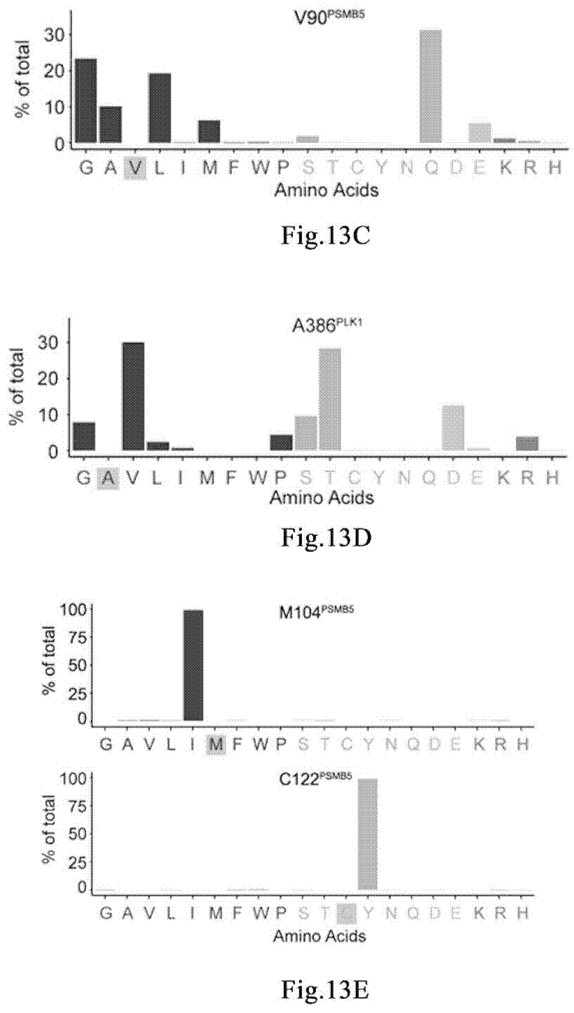

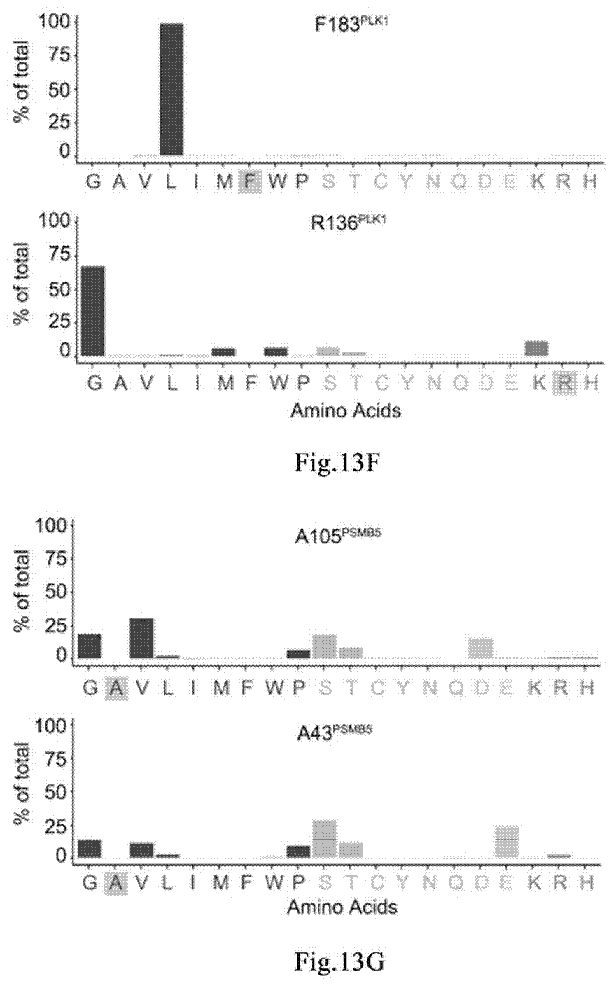

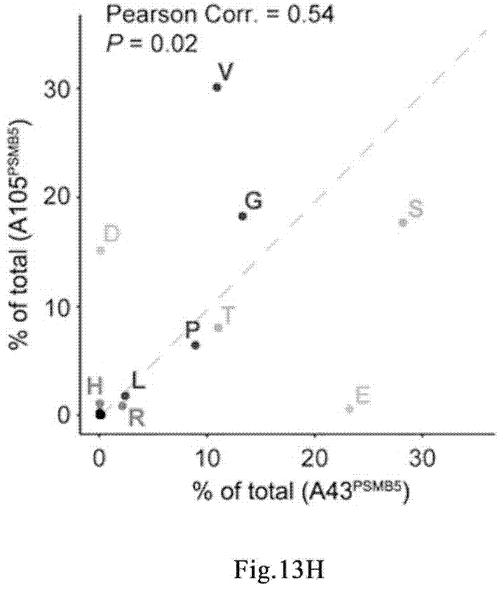

For missense mutation, each amino acid has 19 kinds of nonsynonymous substitutions. We hypothesized that different substitutions might have distinct effects, and some changes might not produce any phenotypic difference. To examine whether CRESMAS strategy could generate such details, we retrieved missense mutation data of top 10 hits from each of PSMB5 and PLK1 screenings, and performed amino acid pattern analysis. We revealed the clear pattern preference for these amino acids, indicating that only certain substitutions could confer cell resistance to drugs (FIG. 13A-B). Multiple substitutions on most sites were capable of evading the deadly effects of drug inhibition, such as V90PSMB5 and A386PLK1 (FIG. 13C-D), whereas only a single specific substitution on some sites could confer drug resistance, such as M104I and C122Y for PSMB5 (FIG. 13E), and F183L for PLK1 (FIG. 13F). R136GPLK1 was not the only mutation type, but the dominant format that conferred cell resistance to BI2536 (FIG. 13F). It was also interesting to notice that two sites in PSMB5, A105 and A43, had very similar mutation preference pattern (FIG. 13G), with a Pearson correlation coefficient of 0.54 (FIG. 13H).

In sum, CRESMAS is a powerful method to generate sequence-to-function maps. It is often very laborious to use truncation mutagenesis to identify potential functional domain, and this becomes increasingly difficult if the protein size is too big. It is also technically difficult, if not impossible, to assess the significance of each and every amino acid spanning the full length of the protein of interest. Gill and colleagues have recently described a method to map functional relevant mutations in protein of interest in bacterium or yeast, however, this method heavily relies on homologous recombination rate, preventing its effective application in higher eukaryotes⁽²⁸⁾. CRESMAS is particularly powerful when dealing with large-sized protein. What's more, one could scan multiple genes simultaneously to obtain functional elements for their corresponding proteins.

The CRISPR saturation mutagenesis provided multiplex mutations covering every amino acid. Different from many other methods, only small percentages of NGS data in respect of in-frame or point mutations were useful reads for CRESMAS. Although we filtered a large number of reads during data preprocessing, we found that our bioinformatics pipeline was sensitive enough to map functional elements from the remaining reads for a moderate sequencing depth. The fact that we could identify most amino acids critical for protein function in all six trials indicates that CRESMAS has low false negative rate.

CRESMAS approach could potentially uncover all residues whose mutations would abolish protein function. However, this does not mean that every hit obtained from CRESMAS screening is directly relevant to protein function. Some residues are important for overall structure of a given protein, but may not directly mediate protein's enzymatic activity or its contact to interaction partner. For instance, we did identify a number of hits located within the transmembrane domain of ANTXR1 (FIG. 5B), a region important to maintain receptor function without direct involvement of toxin endocytosis.

CRESMAS strategy is not limited to only study proteins. It is well suited to acquire functional maps of regulatory elements, such as noncoding RNA, promotors and enhancers. The modification in protocol is to perform PCR amplification on the targeted region on the genome instead of cDNA described above.

### REFERENCES

1. M. Jinek et al., A programmable dual-RNA-guided DNA endonuclease in adaptive bacterial immunity. Science 337, 816-821 (2012).
2. M. E. Burkard, A. Santamaria, P. V. Jallepalli, Enabling and disabling polo-like kinase 1 inhibition through chemical genetics. ACS chemical biology 7, 978-981 (2012).
3. L. Cong et al., Multiplex Genome Engineering Using CRISPR/Cas Systems. Science 339, 819-823 (2013).
4. P. Mali et al., RNA-guided human genome engineering via Cas9. Science 339, 823-826 (2013).
5. O. Shalem et al., Genome-scale CRISPR-Cas9 knockout screening in human cells. Science 343, 84-87 (2014).
6. T. Wang, J. J. Wei, D. M. Sabatini, E. S. Lander, Genetic screens in human cells using the CRISPR-Cas9 system. Science 343, 80-84 (2014).
7. H. Koike-Yusa, Y. Li, E. P. Tan, C. Velasco-Herrera Mdel, K. Yusa, Genome-wide recessive genetic screening in mammalian cells with a lentiviral CRISPR-guide RNA library. Nat Biotechnol 32, 267-273 (2014).
8. Y. Zhou et al., High-throughput screening of a CRISPR/Cas9 library for functional genomics in human cells. Nature 509, 487-491 (2014).
9. G. M. Findlay, E. A. Boyle, R. J. Hause, J. C. Klein, J. Shendure, Saturation editing of genomic regions by multiplex homology-directed repair. Nature 513, 120-123 (2014).
10. M. C. Canver et al., BCL11A enhancer dissection by Cas9-mediated in situ saturating mutagenesis. Nature 527, 192-197 (2015).
11. P. Yuan et al., Chondroitin sulfate proteoglycan 4 functions as the cellular receptor for Clostridium difficile toxin B. Cell Res 25, 157-168 (2015).
12. J. Duan, L. Nilsson, B. Lambert, Structural and functional analysis of mutations at the human hypoxanthine phosphoribosyl transferase (HPRT1) locus. Human mutation 23, 599-611 (2004).
13. M. Steegmaier et al., BI 2536, a potent and selective inhibitor of polo-like kinase 1, inhibits tumor growth in vivo. Curr Biol 17, 316-322 (2007).
14. D. Chen, M. Frezza, S. Schmitt, J. Kanwar, Q. P. Dou, Bortezomib as the first proteasome inhibitor anticancer drug: current status and future perspectives. Curr Cancer Drug Targets 11, 239-253 (2011).
15. M. van Overbeek et al., DNA Repair Profiling Reveals Nonrandom Outcomes at Cas9-Mediated Breaks. Mol Cell 63, 633-646 (2016).
16. S. Zhu et al., Genome-scale deletion screening of human long non-coding RNAs using a paired-guide RNA CRISPR-Cas9 library. Nat Biotechnol 34, 1279-1286 (2016).
17. T. Mitamura et al., Structure-function analysis of the diphtheria toxin receptor toxin binding site by site-directed mutagenesis. J Biol Chem 272, 27084-27090 (1997).
18. S. Fu et al., The structure of tumor endothelial marker 8 (TEM8) extracellular domain and implications for its receptor function for recognizing anthrax toxin. PLoS One 5, e11203 (2010).
19. T. Hart et al., High-Resolution CRISPR Screens Reveal Fitness Genes and Genotype-Specific Cancer Liabilities. Cell 163, 1515-1526 (2015).
20. S. Lu, J. Wang, The resistance mechanisms of proteasome inhibitor bortezomib. Biomark Res 1, 13 (2013).
21. N. E. Franke et al., Impaired bortezomib binding to mutant beta5 subunit of the proteasome is the underlying basis for bortezomib resistance in leukemia cells. Leukemia 26, 757-768 (2012).
22. S. A. Wacker, B. R. Houghtaling, 0. Elemento, T. M. Kapoor, Using transcriptome sequencing to identify mechanisms of drug action and resistance. Nat Chem Biol 8, 235-237 (2012).
23. R. N. Murugan et al., Plkl-targeted small molecule inhibitors: molecular basis for their potency and specificity. Mol Cells 32, 209-220 (2011).
24. C. D. Richardson, G. J. Ray, M. A. DeWitt, G. L. Curie, J. E. Corn, Enhancing homology-directed genome editing by catalytically active and inactive CRISPR-Cas9 using asymmetric donor DNA. Nat Biotechnol, (2016).
25. L. H. de Wilt et al., Proteasome-based mechanisms of intrinsic and acquired bortezomib resistance in non-small cell lung cancer. Biochem Pharmacol 83, 207-217 (2012).
26. E. Suzuki et al., Molecular mechanisms of bortezomib resistant adenocarcinoma cells. PLoS One 6, e27996 (2011).
27. G. T. Hess et al., Directed evolution using dCas9-targeted somatic hypermutation in mammalian cells. Nat Methods, (2016).
28. A. D. Garst et al., Genome-wide mapping of mutations at single-nucleotide resolution for protein, metabolic and genome engineering. Nat Biotechnol 35, 48-55 (2017).

1. A library used for identifying functional elements of a genomic sequence comprising a plurality of CRISPR-Cas system guide RNAs comprising guide sequences that are capable of targeting a plurality of genomic sequences within at least one continuous genomic region, wherein the guide RNAs target at least 100 genomic sequences comprising non-overlapping cleavage sites upstream of a PAM sequence for every 1000 base pairs within the continuous genomic region.

2. The library of claim 1, wherein the library comprises guide RNAs targeting genomic sequences upstream of every PAM sequence within the continuous genomic region.

3. The library of claim 1, wherein each guide RNA is designed to affect about 10 bp around the DSB site.

4. The library according to claim 1, wherein the PAM sequence is specific to at least one Cas protein.

5. The library according to claim 1, wherein the CRISPR-Cas system guide RNAs are selected based upon more than one PAM sequence specific to at least one Cas protein.

6. The library according to claim 1, wherein said targeting results in NHEJ of the continuous genomic region.

7. The library according to claim 1, wherein a cellular phenotype is altered and/or transcription and/or expression of a gene is increased or decreased by said targeting by at least one guide RNA within the plurality of CRISPR-Cas system guide RNAs.

8. The library according to claim 1, which is a plasmid library or viral library.

9. The library according to claim 1, which is a vector library or a host cell library.

10. A method for identifying functional elements of a genomic sequence comprising:
(a) introducing the library of claim 1 into a population of cells that are adapted to contain at least one Cas protein, wherein each cell of the population contains no more than one guide RNA;
(b) sorting the cells into at least two groups based on a change in cellular phenotype;
(c) determining relative representation of the guide RNAs present in each group, whereby genomic sites associated with the change in cellular phenotype are determined by the representation of guide RNAs present in each group;
(d) amplifying one or more cDNA or DNA sequences of the targeted one or more genes for sequencing;
(e) mapping the sequencing reads to reference sequences of the target genes;
(f) filtering the reads to retain those that carry only missense mutations or in-frame deletions; and
(g) determining the weight of each amino acid or nucleotide acid for the cellular phenotype by applying a bioinformatics pipeline.

11. The method of claim 10, wherein the change in cellular phenotype is selected from the group consisting of loss of function, gain of function, decrease of transcription of a gene, increase of transcription of a gene, decrease of expression of a gene and increase of expression of a gene.

12. The method of claim 10, wherein the genomic sequence is for encoding a functional protein.

13. The method of claim 12, which is for identifying functional elements for the protein at single amino acid resolution.

14. The method of claim 10, wherein the genomic sequence is for encoding a non-coding RNA or genetic regulatory element.

15. The method of claim 14, wherein the genetic regulatory element is a promotor or an enhancer.

16. The method of claim 10, wherein the identification is in the native biological context.

17. The method of claim 10, the bioinformatics pipeline comprises:
(h) For fragments containing missense mutations, computing the mutation ratio of each amino acid as follows:
$$mutation ratio = \frac{number of sequence mutations of the amino acid}{total number of sequence reads of the amino acid}$$
(i) For fragments containing in-frame deletions, computing the deletion ratio of each amino acid as follows:
$$deletion ratio = \frac{number of sequence deletions of the amino acid}{total number of sequence reads of the amino acid}$$
(j) Decoding the in-frame deletions and categorizing the in-frame deletions based on the number of amino acid deletions as either “driver deletions”, if they contain only single amino acid deletions, or “passenger deletions”, if they contain multiple amino acid deletions,
(k) Computing the fold changes between the experimental and control groups,
(l) Computing the essential score for each amino acid as follows:
(1) for the mutation fold change, a null distribution is built based on all fold changes, and scoreₘᵤₜₐₜᵢₒₙ=−log10(P-value) is computed for each amino acid,
(2) For the deletion fold change, a tunable parameter, α, is first applied to weight the driver deletion and passenger deletion as follows:
deletion fold change=driver fold change+α*passenger fold change, and then a null distribution is built via permutation 100 times, and score_{deletion}=−log10(P-value) is computed for each amino acid,
(3) scoreₘᵤₜₐₜᵢₒₙ and score_{deletion} are normalized as follows:
$${score}_{mutation} = \frac{( {score}_{mutation} - min ( {score}_{mutation} ) )}{( max ( {score}_{mutation} ) - min ( {score}_{mutation} ) )}$$
$$s c o r {e}_{deletion} = \frac{( scor {e}_{deletion} - min ( scor {e}_{deletion} ) )}{( max ( scor {e}_{deletion} ) - min ( scor {e}_{deletion} ) )}$$
(4) computing the weights of scoreₘᵤₜₐₜᵢₒₙ and score_{deletion} as follows:
$$a = number of amino acids with deletion fold change > 1$$
$$b = number of amino acids with mutation fold change > 1$$
$${w}_{mutation} = \frac{a}{a + b}$$
$${w}_{d e l etion} = \frac{b}{a + b}$$
(5) computing the essential score as follows:
essential score=w_{GHIJIKLM}*score_{GHIJIKLM}+w_{STUTIKLM}*scores_{STUTIKLM};
(6) ranking the amino acids based on their functional importance according to the essential scores.

18. A method of screening functional elements associated with resistance to a drug or toxin comprising:
(a) introducing the library of claim 1 into a population of cells that are adapted to contain a Cas protein, wherein each cell of the population contains no more than one guide RNA;
(b) treating the population of cells with the drug or toxin and sorting the cells into at least two groups based on change in resistance to the drug or toxin;
(c) determining relative representation of the guide RNAs present in each group, whereby genomic sites associated with the change in resistance are determined by the representation of guide RNAs present in each group;
(d) amplifying one or more cDNA or DNA sequences of the targeted one or more genes for sequencing;
(e) mapping the sequencing reads to reference sequences of the target genes;
(f) filtering the reads to retain those that carry only missense mutations or in-frame deletions; and
(g) determining the weight of each amino acid or nucleotide acid for the resistance to the drug or toxin by applying a bioinformatics pipeline.

19. The method of claim 18, wherein the genomic sequence is for encoding a functional protein.

20. The method of claim 19, which is for identifying functional elements for the protein at single amino acid resolution.

21. The method of claim 18, wherein the genomic sequence is for encoding a non-coding RNA or genetic regulatory element.

22. The method of claim 21, wherein the genetic regulatory element is a promotor or an enhancer.

23. The method of claim 18, wherein the identification is in the native biological context.

24. The method of claim 18, wherein the population of cells are introduced into a plurality of guide RNAs comprising guide sequences that are capable of targeting a plurality of genomic sequences within at least one continuous genomic region, wherein the guide RNAs target at least 100 genomic sequences comprising non-overlapping cleavage sites upstream of a PAM sequence for every 1000 base pairs within the continuous genomic region.

25. The method of claim 24, wherein each guide RNA is designed to affect about 10 bp around the DSB site.

26. The method of claim 24, wherein the PAM sequence is specific to at least one Cas protein.

27. The method of claim 24, wherein the CRISPR-Cas system guide RNAs are selected based upon more than one PAM sequence specific to at least one Cas protein.

28. The method of claim 18, the bioinformatics pipeline comprises:
(h) For fragments containing missense mutations, computing the mutation ratio of each amino acid as follows:
$$mutation ratio = \frac{number of sequence mutations of the amino acid}{total number of sequence reads of the amino acid}$$
For fragments containing in-frame deletions, computing the deletion ratio of each amino acid as follows:
$$deletion ratio = \frac{number of sequence deletions of the amino acid}{total number of sequence reads of the amino acid}$$
(j) Decoding the in-frame deletions and categorizing the in-frame deletions based on the number of amino acid deletions as either “driver deletions”, if they contain only single amino acid deletions, or “passenger deletions”, if they contain multiple amino acid deletions,
(k) Computing the fold changes between the experimental and control groups,
(l) Computing the essential score for each amino acid as follows:
(1) for the mutation fold change, a null distribution is built based on all fold changes, and scoreₘᵤₜₐₜᵢₒₙ=−log10(P-value) is computed for each amino acid,
(2) the deletion fold change, a tunable parameter, α, is first applied to weight the driver deletion and passenger deletion as follows:
deletion fold change=driver fold change+α*passenger fold change, and then a null distribution is built via permutation 100 times, and scoreddetton=−log10(P-value) is computed for each amino acid,
(3) scoreₘᵤₜₐₜᵢₒₙ and score_{delection} are normalized as follows:
$${score}_{mutation} = \frac{( {score}_{mutation} - min ( {score}_{mutation} ) )}{( max ( {score}_{mutation} ) - min ( {score}_{mutation} ) )}$$
$$s c o r {e}_{deletion} = \frac{( scor {e}_{deletion} - min ( scor {e}_{deletion} ) )}{( max ( scor {e}_{deletion} ) - min ( scor {e}_{deletion} ) )}$$
(4) computing the weights of scoreₘᵤₜₐₜᵢₒₙ and score_{delection} as follows:
$$a = number of amino acids with deletion fold change > 1$$
$$b = number of amino acids with mutation fold change > 1$$
$${w}_{mutation} = \frac{a}{a + b}$$
$${w}_{d e l etion} = \frac{b}{a + b}$$
(5) computing the essential score as follows:
essential score=w_{GHIJIKLM}*score_{GHIJIKLM}+w_{STUTIKLM}*scores_{STUTIKLM};
(6) ranking the amino acids based on their functional importance according to the essential scores.

29. A method for identifying functional elements for a protein of interest comprising conducting saturation mutagenesis to the protein of interest by disrupting the genomic gene coding for the protein by using CRISPR-Cas system introduced into a population of cells, determining disrupted genomic sites associated with change of phenotype by sequencing DNA and cDNA of the targeted gene, retrieving in-frame mutations that give rise to the change of phenotype, and building a bioinformatics pipeline to identify functional elements of the protein of interest at single amino acid resolution.

30. The method of claim 29, wherein the identification of the functional elements for the protein of interest is in its native biological context.

31. The method of claim 29, wherein the in-frame mutations are in-frame deletions and missense point mutations.

32. The method of claim 29, wherein the change in cellular phenotype is selected from the group consisting of loss of function, gain of function, decrease of transcription of a gene, increase of transcription of a gene, decrease of expression of a gene and increase of expression of a gene.

33. The method of claim 29, which is for identifying functional elements for the protein at single amino acid resolution.

34-36. (canceled)

37. The method of claim 29, wherein each cell of the population contains no more than one guide RNA, and a plurality of guide RNAs introduced to the population of cells comprise guide sequences that are capable of targeting a plurality of genomic sequences within at least one continuous genomic region coding for the protein of interest, wherein the guide RNAs target at least 100 genomic sequences comprising non-overlapping cleavage sites upstream of a PAM sequence for every 1000 base pairs within the continuous genomic region.

38. The method of claim 37, wherein each guide RNA is designed to affect about 10 bp around the DSB site.

39. The method of claim 37, wherein the PAM sequence is specific to at least one Cas protein.

40. The method of claim 29, wherein the CRISPR-Cas system guide RNAs are selected based upon more than one PAM sequence specific to at least one Cas protein.

41. The method of claim 29, wherein the bioinformatic pipeline comprises:
Mapping sequencing reads to the reference sequences of the target gene by using bioinformatic tools,
Filtering the reads to retain those that carried only missense mutations or in-frame deletions,
For fragments containing missense mutations, computing the mutation ratio of each amino acid as follows:
$$mutation ratio = \frac{number of sequence mutations of the amino acid}{total number of sequence reads of the amino acid}$$
ii) For fragments containing in-frame deletions, computing the deletion ratio of each amino acid as follows:
$$deletion ratio = \frac{number of sequence deletions of the amino acid}{total number of sequence reads of the amino acid}$$
ii) Decoding the in-frame deletions and categorizing the in-frame deletions based on the number of amino acid deletions as either “driver deletions”, if they contain only single amino acid deletions, or “passenger deletions”, if they contain multiple amino acid deletions,
iii) Computing the fold changes between the experimental and control groups,
iv) Computing the essential score for each amino acid as follows:
(1) for the mutation fold change, a null distribution is built based on all fold changes, and scoreₘᵤₜₐₜᵢₒₙ=−log10(P-value) was computed for each amino acid,
(2) For the deletion fold change, a tunable parameter, α, is first applied to weight the driver deletion and passenger deletion as follows:
deletion fold change=driver fold change+α*passenger fold change, and then a null distribution is built via permutation 100 times, and score_{deletion}=−log10(P-value) is computed for each amino acid,
(3) scoremutation and scoreddetion are normalized as follows:
$${score}_{mutation} = \frac{( {score}_{mutation} - min ( {score}_{mutation} ) )}{( max ( {score}_{mutation} ) - min ( {score}_{mutation} ) )}$$
$$s c o r {e}_{deletion} = \frac{( scor {e}_{deletion} - min ( scor {e}_{deletion} ) )}{( max ( scor {e}_{deletion} ) - min ( scor {e}_{deletion} ) )}$$
(4) computing the weights of scoremutation and scoreddetion as follows:
$$a = number of amino acids with deletion fold change > 1$$
$$b = number of amino acids with mutation fold change > 1$$
$${w}_{mutation} = \frac{a}{a + b}$$
$${w}_{d e l etion} = \frac{b}{a + b}$$
(5) computing the essential score as follows:
essential score=w_{GHIJIKLM}*score_{GHIJIKLM}+w_{STUTIKLM}*scores_{STUTIKLM};
(6) ranking the amino acids based on their functional importance according to the essential scores.

42. (canceled)
